(19) United States
(12) Patent Application Publication
(10) Pub. No.: US 2004/0167067 A1
(43) Pub. Date: Aug. 26, 2004

(54) ESM-1 gene differentially expressed in angiogenesis, antagonists thereof, and methods of using the same

(76) Inventors: David Griggs, Ballwin, MO (US); Richard Head, Florissant, MO (US); Richard Mazzarella, Webster Groves, MO (US); Edward Weinstein, Lansdale, PA (US)

Correspondence Address: HARNESS, DICKEY, & PIERCE, P.L.C, 7700 BONHOMME, STE 400, ST. LOUIS, MO, 63105, US

(21) Appl. No.: 10/611,527

(22) Filed: Jul. 1, 2003

Related U.S. Application Data
(60) Provisional application No. 60/392,784, filed on Jul. 1, 2002.

(51) Int. Cl. A61K 38/17; C07K 14/705; C07H 21/04

(52) U.S. Cl. 514/12; 530/350; 435/69.1; 435/320.1; 435/325; 536/23.5

(57) ABSTRACT

The invention relates a nucleic acid and its encoded polypeptide ESM-1, whose expression is modulated in angiogenesis and oncogenesis. The invention also relates to antibodies having specificity for said polypeptide. The present invention also relates to antisense molecules. The invention further relates to methods useful for treating or modulating angiogenesis in mammals in need of such biological effect.

The present application claims priority under Title 35, United States Code, §119 to United States Provisional application Serial No. 60/392,784, filed Jul. 1, 2002, which is incorporated by reference in its entirety as if written herein.

### FIELD OF THE INVENTION

The invention relates generally to the identification of a nucleic acid and its encoded polypeptides, whose expression is modulated in angiogenesis and oncogenesis. The invention also relates to antibodies having specificity for said polypeptide. The present invention also relates to antisense molecules. The invention further relates to methods useful for treating or modulating angiogenesis in mammals in need of such biological effect. This includes the diagnosis and treatment of angiogenic disorders including, but not limited to, wound healing and cancer. Additionally, the present invention further relates to the use of antibodies against the polypeptides of the present invention as diagnostic probes or as therapeutic agents as well as the use of polynucleotide sequences encoding the polypeptides of the present invention as diagnostic probes or therapeutic agents for the treatment of broad range of pathological states including cardiovascular, angiogenic, oncological, and endothelial disorders.

### BACKGROUND OF THE INVENTION

Angiogenesis is the growth of new capillary blood vessels from pre-existing vessels and capillaries and is crucial in a large number of processes, such as wound repair, embryonic development, and the growth of solid tumors. In neovascularization, endothelial cells will undergo migration, elongation, proliferation, and orientation leading to lumen formation, re-establishment of a basement membrane and eventual anastomosis with other vessels (Patan, 2000).

Endothelial cell-specific molecule1 (ESM-1) was originally isolated in an immuno screening of a HUVEC cDNA library in order to identify the gene encoding a 55-kDa autoantigen that may have a role in asthma (Lassalle, et al., 1993). The full length ESM-1 cDNA was cloned in a library constructed in pCDM8 but was found to be inserted in the reverse orientation (Lassalle et al., 1996).

Northern blots have shown ESM-1 to probes to hybridize to RNA from HUVEC cells, SV40-transfected HUVECs, human lung, and human kidney. Little or none was detected in human heart, pancreas, placenta, muscle, brain, or liver (Lassalle et al., 1996). Antibodies raised to ESM-1 show protein expression in human lung, colon, and kidney (Bechard et al., 2000; Lassalle et al., WO9945028). In the lung, ESM-1 is expressed in venules, arterioles, and alveolar capillaries as well as by epithelial cells of the bronchi and submucosal glands. In the kidney, expression is predominantly in renal tubular epithelial cells. Capillaries and venules of the lamina propria of the colon also display ESM-1 expression. A splice variant of ESM-1 has been identified which lacks 150 base pairs but maintains the open reading frame (Aitkenhead et al., 2002).

WO 99/45028 discloses monoclonal antibodies to ESM-1 and the use of the antibody to detect ESM-1. WO 02/39123 discloses a kit comprising an antibody specifically binding the N-terminal region of ESM-1 and a second antibody specifically binding C-terminal region of ESM-1. WO 02/38178 discloses antagonistic antibodies to ESM for the treatment of cancer.

### SUMMARY OF THE INVENTION

In one aspect, the invention involves a method of assessing the efficacy of an angiogenic disorder treatment in a subject, wherein the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose cancerous stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian subject. The expression of the nucleic acid sequences may be either increased or decreased in the test cell population as compared to the reference cell population.

In a further aspect, the invention involves a method of diagnosing an angiogenic disorder, wherein the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose angiogenesis stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian subject. The expression of the nucleic acid sequences may be either increased or decreased in the test cell population as compared to the reference cell population.

In another aspect, the invention involves a method of identifying a test therapeutic agent for treating an angiogenic disorder in a subject involving the steps of providing a test cell population capable of expressing one or more of the nucleic acid sequences of the present invention; contacting the test cell population with the test therapeutic agent; detecting the expression of one or more of these nucleic acid sequences; comparing the expression to that of the nucleic acid sequences in a reference cell population whose angiogenesis stage is known; and identifying a difference in expression level, if present, between the test cell population and the reference cell population. In different embodiments, the subject may be a mammal or, more preferably, a human. Additionally, the test therapeutic agent may be either a known angiogenic disorder agent or an unknown angiogenic disorder agent. The antagonist may be an antibody having selectivity to at least one of the polypeptides of the present invention. The angiogenic disorder to be treated can be selected from the following diseases or disorders: breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendrogliorna, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

In a further aspect, the invention involves a method of identifying or determining the susceptibility to, predisposition to, or presence of, an angiogenic disorder in a subject. In this aspect, the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose cancerous stage is known; and identifying a difference in expression level, if present, between the test cell population and the reference cell population. The subject may be a mammal, or, more preferably, a human.

In an alternative aspect, the invention involves a method of treating an angiogenic disorder by administering an agent that modulates the expression or activity of one or more of the nucleic acid sequences of the present invention to a patient suffering from or at risk for developing the angiogenic disorder. This agent can be one that decreases the expression of one or more of sequences of the present invention that are up regulated in cancerous tissues. Alternatively, it can be one that increases the expression of one or more of sequences of the present invention that are down regulated. Additionally, the agent can be an antibody to a polypeptide encoded by the nucleic acid sequence, an antisense nucleic acid molecule, a peptide, a polypeptide agonist, a polypeptide antagonist, a peptidomimetic, a small molecule, or another drug.

The present invention is directed to antisense compounds, particularly oligonucleotides, which are targeted to a nucleic acid encoding ESM-1, and which modulate the expression of ESM-1. Pharmaceutical and other compositions comprising the antisense compounds of the invention are also provided. Further provided are methods of modulating the expression of ESM-1 in cells or tissues comprising contacting said cells or tissues with one or more of the antisense compounds or compositions of the invention. Further provided are methods of treating an animal, particularly a human, suspected of having or being prone to a disease or condition associated with expression of ESM-1 by administering a therapeutically or prophylactically effective amount of one or more of the antisense compounds or compositions of the invention.

The invention also includes a kit containing one or more reagents for detecting two or more of the nucleic acid sequences of the present invention. Additionally, the invention involves an array of probe nucleic acids capable of detected two or more of the nucleic acids of the present invention.

The polypeptides, nucleic acids, and antibodies of the invention can be used to treat an angiogenic disorder in a subject. Treatment of an angiogenic disorder may be in a mammal, preferably a human. In various embodiments, therapeutic compositions containing the polypeptides and nucleic acids of the invention can be used to treat angiogenic disorders. These therapeutic compositions can include a pharmaceutically acceptable carrier and, additionally, an active ingredient such as an anti-angiogenesis agent or an anti-inflammatory agent. Also provided is a kit containing a therapeutic composition for use in the treatment of an angiogenic disorder along with a pharmaceutically acceptable carrier, wherein the therapeutic composition is a polypeptide of the present invention, an agonist of a polypeptide of the present invention, or an antagonist of a polypeptide of the present invention.

Also included in the invention is an isolated nucleic acid molecule that is at least 80% identical to the nucleic acid encoding the polypeptide of the present invention or the complement of the nucleic acid sequence, as well as vectors and host cells containing this nucleic acid sequence. Also provided is a method for producing a polypeptide by culturing a host cell transformed with one or more vectors described herein under conditions suitable for the expression of the protein encoded by the vector.

In another aspect, there is provided an isolated polypeptide encoded by an isolated nucleic acid sequence or oligonucleotide described herein. In some aspects, the isolated protein, functional variants or fragments thereof. In another embodiment, a variant or fragment of a protein of the present invention retains the respective activity.

A further embodiment of the present invention is markers and methods for assessing the efficacy of anti angiogenesis treatments based on monitoring the level of a nucleic acid or polypeptide of the present invention in a biological sample.

Other features and advantages of the invention will be apparent from the following detailed description and from the claims.

### BRIEF DESCRIPTION OF THE FIGS.

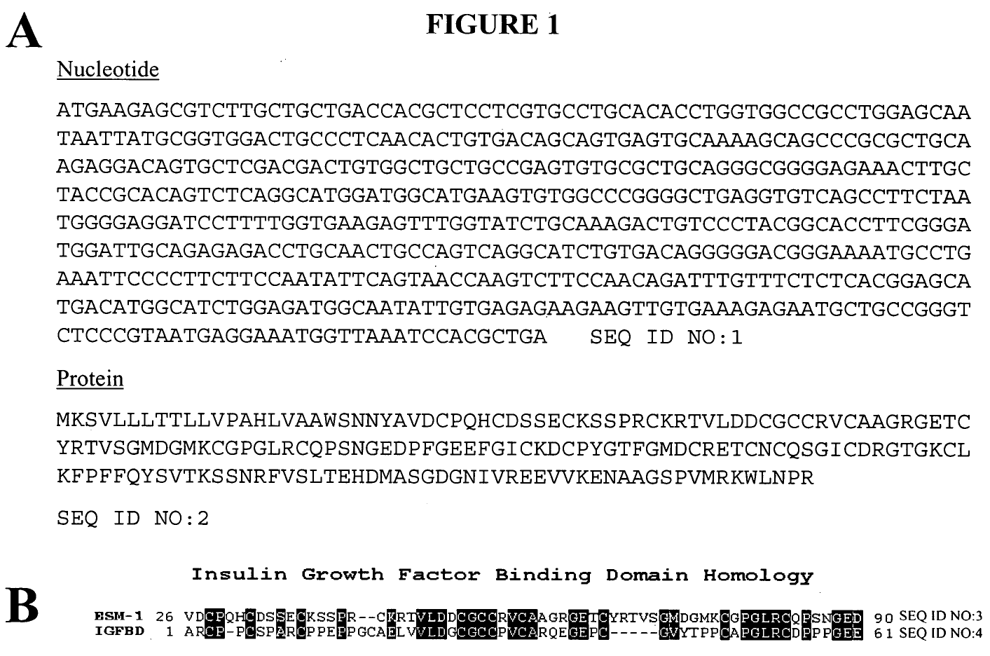

FIG. 1. Human ESM-1 nucleotide and amino acid sequence. A. Nucleotide sequence of the coding region of human ESM-1 cDNA, SEQ ID NO:1, and the deduced protein sequence, SEQ ID NO:2. B Alignment of amino acids 26-90, SEQ ID NO:3, of the human ESM-1 protein, to the consensus insulin growth factor binding domain, SEQ ID NO:4. Identical amino acids between the human ESM-1 protein and the domain are indicated by black boxes whereas synonymous amino acids are indicated in gray. Gaps inserted in the sequences to obtain maximum homology are shown as dashes.

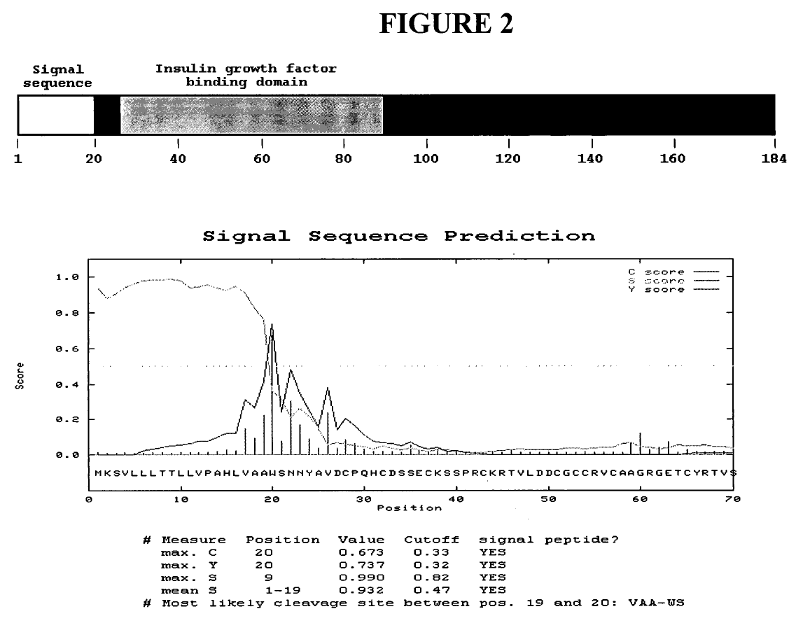

FIG. 2. Functional domains of the human ESM-1 protein. The human ESM-1 protein is depicted as a linear bar (top) with the relative position of the signal sequence shown in white and of the insulin growth factor binding domain shown in gray. Amino acid number is labeled at the bottom of the bar. Results of the first 70 amino acids of the human ESM-1 in the SignalP analysis are also shown (bottom).

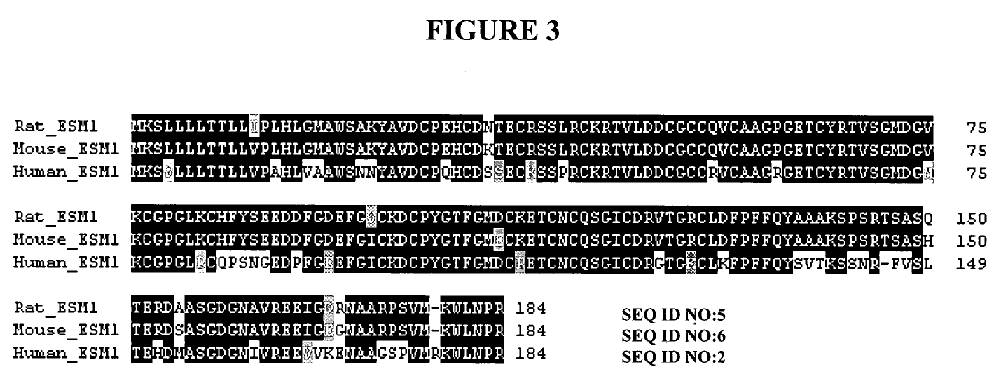

FIG. 3. Comparison of ESM-1 orthologs. A multiple alignment of the human (SEQ ID NO:2), mouse (SEQ ID NO:6) and rat (SEQ ID NO:5) ESM-1 proteins is shown. Identical residues in two or more of the proteins are illustrated by black boxes and conservative amino acid changes are illustrated in gray. Gaps introduced into sequences to obtain the best multiple alignment are shown as dashes.

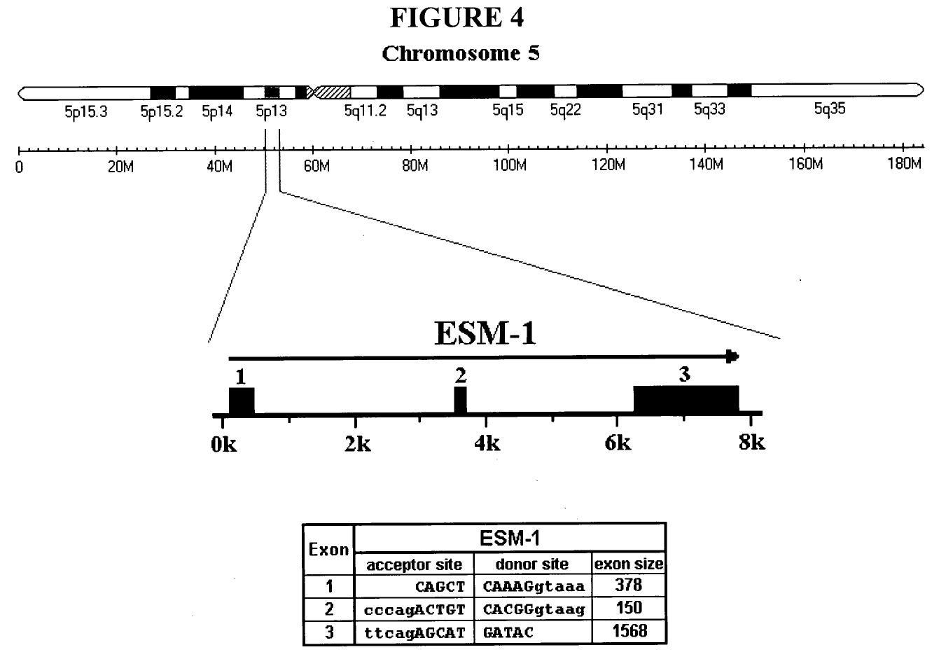

FIG. 4. Human genome location and genomic structure of the ESM-1 gene. Chromosome 5p13 contains the ESM-1 gene, which encoded by 3 exons, spanning about 8 kb, transcribed in the telomeric to centromeric direction.

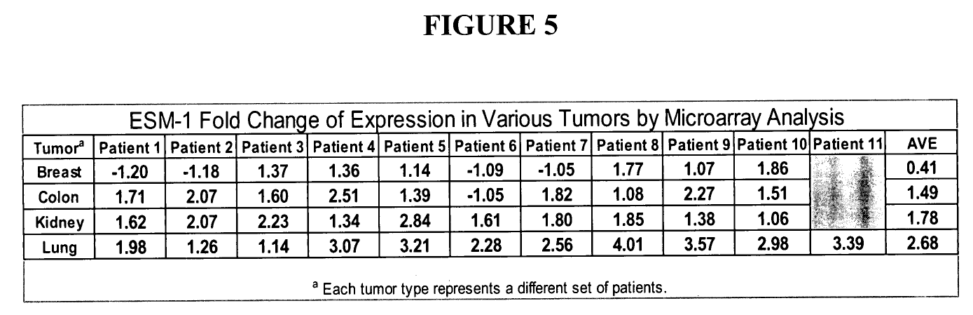

FIG. 5. Changes in the levels of expression of ESM-1 in tumors from various patients measured by microarray analysis. The fold change in expression was determined by comparing tumors from patients having breast, colon, and kidney or lung carcinoma against normal tissue from the same organ. Changes >2-fold are considered to be significant since they have a high probability (>95%) of confirming a >2-fold change by subsequent more quantitative measurements.

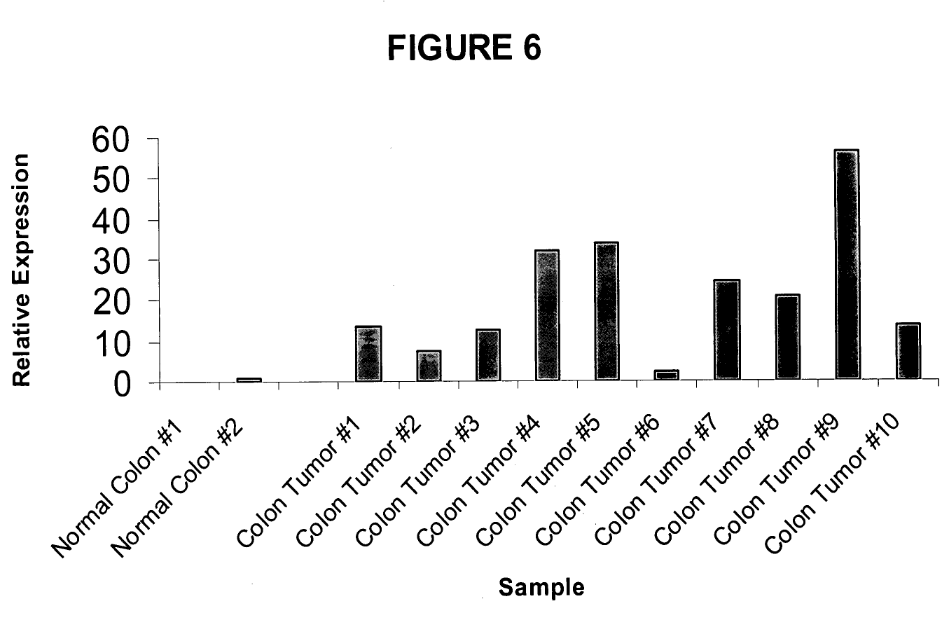

FIG. 6. Relative expression of humane ESM-1 in colon tumors. Quantitative real time reverse transcription polymerase chain reaction performed on tumors and non-involved tissue. Expression of ESM-1 is measured and normalized to expression of the housekeeping gene GUS. The level of ESM-1 expression for the normal colon tissue #1 is then designated as having a relative expression value of 1.

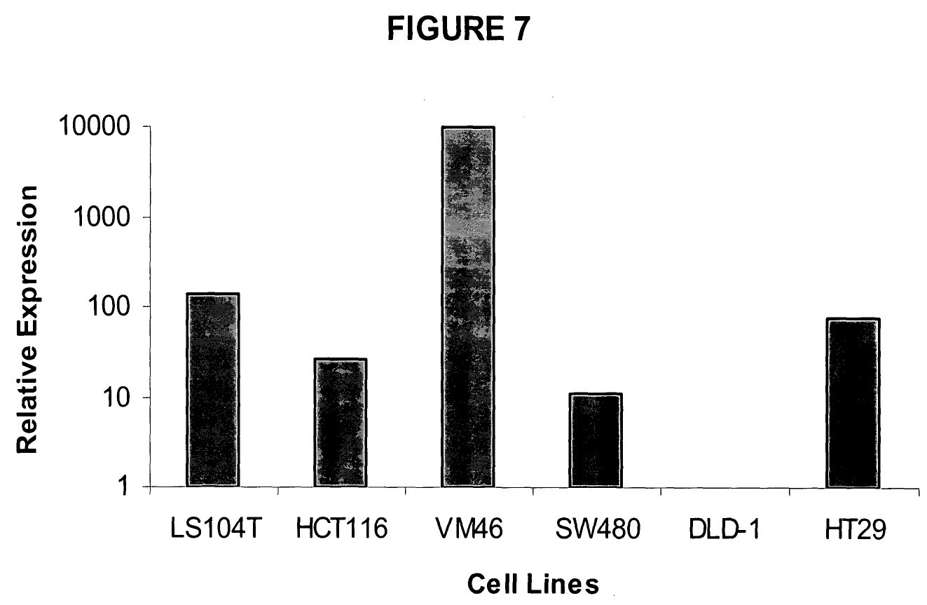

FIG. 7. Expression of ESM-1 in human colon carcinoma cells in culture. Quantitative real time reverse transcription polymerase chain reaction performed on colon carcinoma cells in culture. Expression of ESM-1 is measured in all samples and normalized to expression of the housekeeping gene cyclophilin. The value of the level of ESM-1 for the lowest expressing cell line, DLD-1, is designated as having a relative expression of 1.

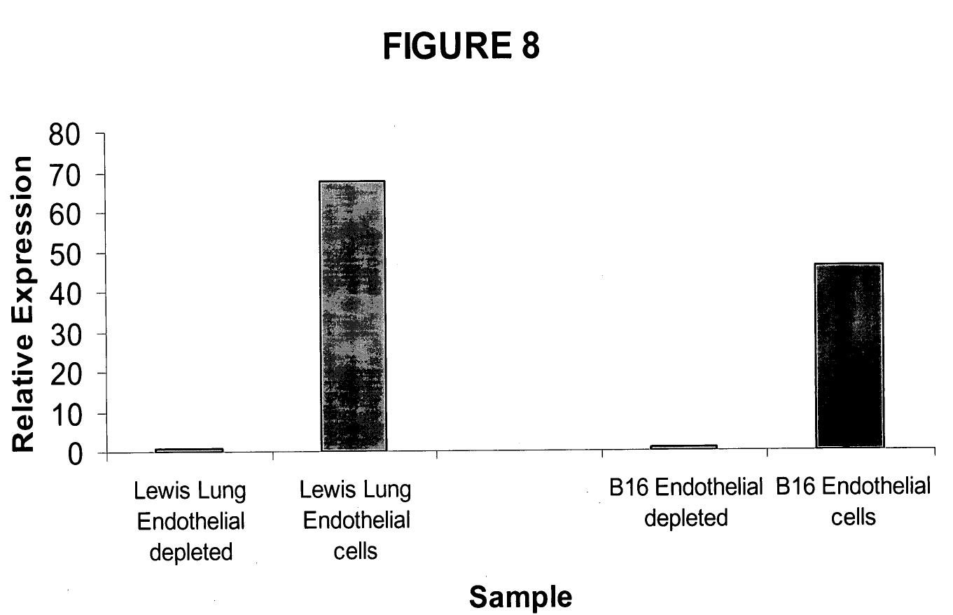

FIG. 8. Expression of ESM-1 in tumor enriched and depleted endothelial cells derived from murine in vivo tumor models. Quantitative real time reverse transcription polymerase chain reaction performed on RNA from tumor endothelial cells and epithelial tumor cells depleted of endothelial cells. Expression of ESM-1 is measured in all samples and normalized to the expression of the housekeeping gene GUS. The value for the level of ESM-1 expression in the Lewis Lung endothelial-depleted population is designated as 1, and the value for the level of ESM-1 expression in Lewis Lung endothelial cells is then compared to that of the endothelial-depleted population. Similarly, the value for the level of ESM-1 expression in the B16 melanoma endothelial-depleted population is designated as 1 for comparison to the level of ESM-1 in the B16 melanoma endothelial cell population.

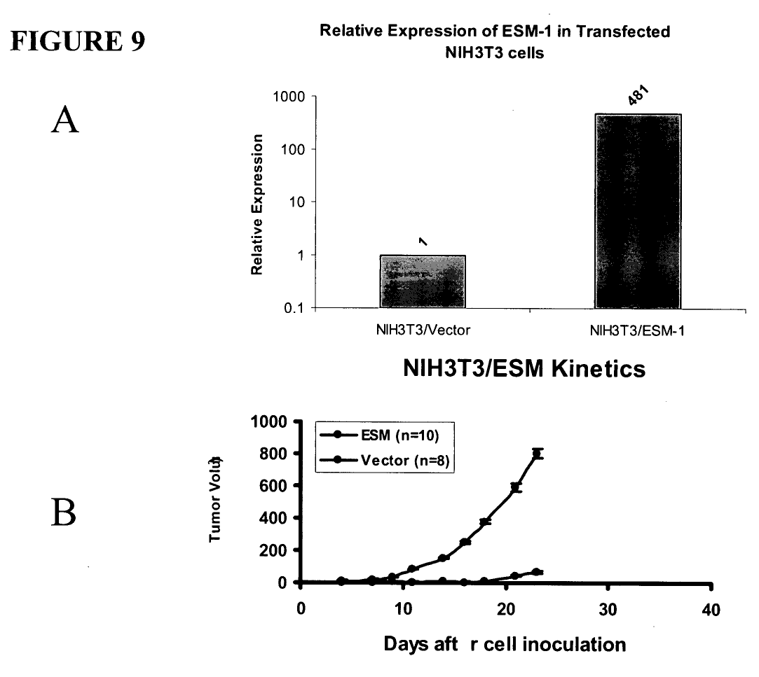

FIG. 9. NIH3T3 cell expression of ESM-1 and transformation in nu/nu female mice. NIH3T3 cells transfected with vector or ESM-1 gene under control of CMV promoter were assayed for levels of ESM-1 RNA by quantitative real time reverse transcription polymerase chain reaction. Expression of ESM-1 was normalized to expression of the housekeeping gene cyclophilin. The value for the level of ESM-1 expression in the vector-transfected cells was designated as 1, and the value for the level of ESM-1 expression in the ESM-1 transfected cells were then compared to it. The relative expression levels are noted above bars. Tumor volumes were measured in each animal over the course of three weeks.

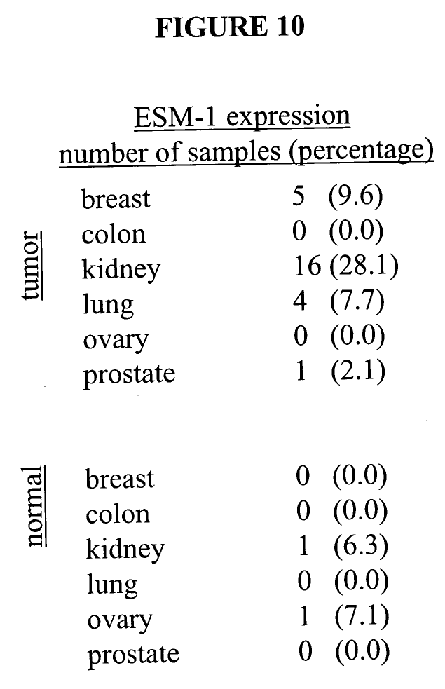

FIG. 10. In situ hybridization (ISH) results for ESM-1 on a variety of tumor samples. ³³P-labeled oligo probe cocktail shows ESM-1 is expressed in 28.1% of renal cell carcinomas, 9.6% of breast carcinomas, and 7.7% of lung carcinomas.

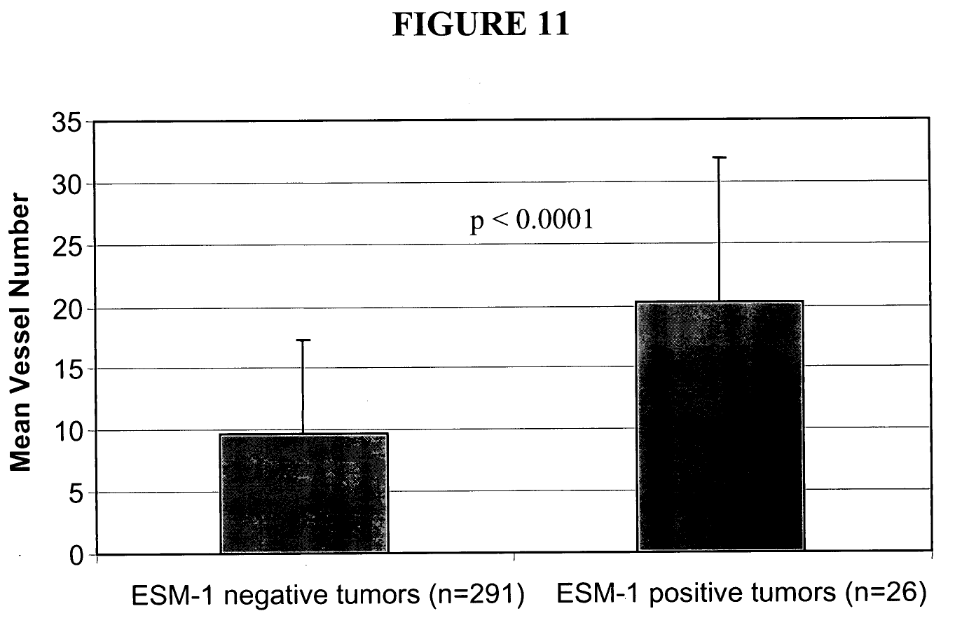

FIG. 11. Correlation of ESM-1 expression to blood vessel density. Immunohistochemical staining with Factor VIII antibody on 317 tumor samples was performed (colon, prostate, lung, breast, renal, and ovary) and compared to ESM-1 expression as determined by ISH. ESM-1 positive tumors display a significantly higher mean number of blood vessels than do ESM-1 negative tumors.

### DETAILED DESCRIPTION OF THE INVENTION

Angiogenesis is the development of new blood vessels from existing capillaries. This process has been implicated in a number of human diseases, as well as in the growth and metastasis of solid tumors. In some forms of arthritis, new capillaries form in the joint, leading to its gradual destruction. Solid tumors also must stimulate the formation of new blood vessels in order to obtain the nutrients and oxygen necessary for their growth, thus providing a route by which the tumors can metastasize to distant sites.

Experimental evidence has suggested that malignant tumors can induce angiogenesis through the elaboration of a variety of factors, such as acidic fibroblast growth factor (aFGF), basic fibroblast growth factor (bFGF), vascular endothelial growth factor (VEGF); platelet derived growth factor (PDGF), transforming growth factor-alpha (TNF-alpha), and many others (Liotta et al., 1991, Cell 64: 327-336; Hanahan et al., Cell 86: 353-364).

As used herein, the term “angiogenesis” means the generation of new blood vessels into a tissue or organ, and involves endothelial cell proliferation. Under normal physiological conditions, humans or animals undergo angiogenesis only in very specific restricted situations. For example, angiogenesis is normally observed in wound healing, fetal and embryonal development, and formation of the corpus luteum, endometrium, and placenta. The term “endothelium” means a thin layer of flat epithelial cells that lines serous cavities, lymph vessels, and blood vessels.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic dependent or angiogenic associated diseases.

The hypothesis that tumor growth is angiogenesis-dependent was first proposed in 1971. (Folkman J., N. Engl. Jour. Med. 285:1182 1186, 1971) In its simplest terms it states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:

The growth rate of tumors implanted in subcutaneous transparent chambers in mice is slow and linear before neovascularization, and rapid and nearly exponential after neovascularization. (Algire G H, et al. Vascular reactions of normal and malignant tumors in vivo. I. Vascular reactions of mice to wounds and to normal and neoplastic transplants. J. Natl. Cancer Inst. 6:73-85, 1945)

Tumors grown in isolated perfused organs where blood vessels do not proliferate are limited to 1-2 mm³ but expand rapidly to >1000 times this volume when they are transplanted to mice and become neovascularized. (Folkman J, et al., Tumor behavior in isolated perfused organs: In vitro growth and metastasis of biopsy material in rabbit thyroid and canine intestinal segments. Annals of Surgery 164:491-502, 1966)

Tumor growth in the avascular cornea proceeds slowly and at a linear rate, but switches to exponential growth after neovascularization. (Gimbrone, M. A., Jr. et al., Tumor growth and neovascularization: An experimental model using the rabbit cornea. J. Nail. Cancer Institute 52:41-427, 1974)

Tumors suspended in the aqueous fluid of the anterior chamber of the rabbit eye, remain viable, avascular and limited in size to <1 mm³. Once they are implanted on the iris vascular bed, they become neovascularized and grow rapidly, reaching 16,000 times their original volume within 2 weeks. (Gimbrone M A Jr., et al., Tumor dormancy in vivo by prevention of neovascularization. J. Exp. Med. 136:261-276)

When tumors are implanted on the chick embryo chorioallantoic membrane, they grow slowly during an avascular phase of >72 hours, but do not exceed a mean diameter of 0.93-0.29 mm. Rapid tumor expansion occurs within 24 hours after the onset of neovascularization, and by day 7 these vascularized tumors reach a mean diameter of 8.0+2.5 mm. (Knighton D., Avascular and vascular phases of tumor growth in the chick embryo. British J. Cancer, 35:347-356, 1977)

Vascular casts of metastases in the rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien W., et al., The blood supply of experimental liver metastases. II. A microcirculatory study of normal and tumor vessels of the liver with the use of perfused silicone rubber. Surgery 68:334-340, 1970)

In transgenic mice, which develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman J, et al., Induction of angiogenesis during the transition from hyperplasia to neoplasia. Nature 339:58-61, 1989)

A specific antibody against VEGF (vascular endothelial growth factor) reduces microvessel density and causes “significant or dramatic” inhibition of growth of three human tumors, which rely on VEGF as their sole mediator of angiogenesis (in nude mice). The antibody does not inhibit growth of the tumor cells in vitro. (Kim K J, et al., Inhibition of vascular endothelial growth factor-induced angiogenesis suppresses tumor growth in vivo. Nature 362:841-844, 1993)

Anti-bFGF monoclonal antibody causes 70% inhibition of growth of a mouse tumor, which is dependent upon secretion of bFGF as its only mediator of angiogenesis. The antibody does not inhibit growth of the tumor cells in vitro. (Hori A, et al., Suppression of solid tumor growth by immunoneutralizing monoclonal antibody against human basic fibroblast growth factor. Cancer Research, 51:6180-6184, 1991)

Intraperitoneal injection of bFGP enhances growth of a primary tumor and its metastases by stimulating growth of capillary endothelial cells in the tumor. The tumor cells themselves lack receptors for bFGF, and bFGF is not a mitogen for the tumors cells in vitro. (Gross J L, et al. Modulation of solid tumor growth in vivo by bFGF. Proc. Amer. Assoc. Canc. Res. 31:79, 1990)

A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber D, et al., Angioinhibins: Synthetic analogues of fumagillin which inhibit angiogenesis and suppress tumor growth. Nature, 48:555-557, 1990). There is also indirect clinical evidence that tumor growth is angiogenesis dependent.

Human retinoblastomas that are metastatic to the vitreous develop into avascular spheroids, which are restricted to less than 1 mm³ despite the fact that they are viable and incorporate 3H-thymidine (when removed from an enucleated eye and analyzed in vitro).

Carcinoma of the ovary metastasizes to the peritoneal membrane as tiny avascular white seeds (1-3 mm³). These implants rarely grow larger until one or more of them become neovascularized.

Intensity of neovascularization in breast cancer (Weidner N., et al., Tumor angiogenesis correlates with metastasis in invasive breast carcinoma. N. Engl. J. Med. 324:1-8, 1991, and Weidner N., et al., Tumor angiogenesis: A new significant and independent prognostic indicator in early-stage breast carcinoma, J Natl. Cancer Inst. 84:1875-1887, 1992) and in prostate cancer (Weidner N, Carroll P R, Flax J, Blumenfeld W, Folkman J. Tumor angiogenesis correlates with metastasis in invasive prostate carcinoma. American Journal of Pathology, 143(2): 401-409, 1993) correlates highly with risk of future metastasis.

Metastasis from human cutaneous melanoma is rare prior to neovascularization. The onset of neovascularization leads to increased thickness of the lesion and an increasing risk of metastasis. (Srivastava A, et al., The prognostic significance of tumor vascularity in intermediate thickness (0.76-4.0 mm thick) skin melanoma. Amer. J. Pathol. 133:419-423, 1988)

In bladder cancer, the urinary level of an angiogenic peptide, bFGF, is a more sensitive indicator of status and extent of disease than is cytology. (Nguyen M, et al., Elevated levels of an angiogenic peptide, basic fibroblast growth factor, in urine of bladder cancer patients. J. Natl. Cancer Inst. 85:241-242, 1993)

Thus, it is clear that angiogenesis plays a major role in the metastasis of a cancer. If this angiogenic activity could be repressed or eliminated, or otherwise controlled and modulated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

ESM-1 expression appears to be both constitutive and under the control of a variety of cytokines. HUVEC cells treated with TNFα or IL-1β display an up-regulation of the gene. No change in ESM-1 levels was seen upon treatment with IL-4 or IFNγ. While coadministration of TNF□ and IFNγ lead to a synergistic induction of proinflammatory factors such as IL-6, IL-8, RANTES and ICAM-1, the combination of these two cytokines inhibit the TNFα induced ESM-1 up-regulation (Lassale et al., 1996).

ESM-1 was amplified from HDMECs and cloned it into an expression vector. A pool of transfected NIH3T3 cells were then selected and assayed for ESM-1 expression. After confirming significant gene over-expression at the RNA level, cells were injected subcutaneously into a nu/nu female mouse. While vector transfected NIH3T3 fibroblasts failed to grow in these mice, those cells transfected with ESM-1 formed solid tumors within three weeks. This data shows that ESM-1 contains the potential to augment growth in vivo to a cell line that is usually not capable of forming tumors.

ESM-1 gene expression has been shown to change in cells under varying conditions. These findings have been extended to show that ESM-1 is up-regulated in colon carcinomas when compared to normal colon tissue. Additionally, it is shown that forced over-expression of ESM-1 leads to an escalation of growth of NIH3T3 fibroblasts in vivo.

ESM-1 over-expression in three independent Incyte microarray experiments showed selective expression in cultured HUVEC cells versus pooled human tissue (28.8 and 13.3-fold). Another Incyte microarray experiment showed higher expression (>10-fold) in HMVEC cells growing on collagen relative to those growing on osteopontin. ESM-1 has also been found to be differentially expressed in endothelial cells forming tubes in a 3-dimensional collagen gel when compared to cells growing in two dimensions (Aitkenhead et al., 2002).

In addition to endothelial expression, ESM-1 is over-expressed in a significant number of colon tumors when compared to a pool of normal colon tissue. Nine of ten tumors showed expression at levels of threefold or higher at the RNA level, as determined by real-time quantitative reverse transcription polymerase chain reaction experiments. ESM-1 has also been shown to be over-expressed in renal cell carcinoma (5/14) (Aitkenhead et al., 2002).

In order to determine transcripts that are up-regulated in HMVECs when grown on an angiogenesis stimulating matrix, microarray analysis was performed on Incyte DNA chips containing cDNAs representing approximately 30,000 genes. Isolated RNA samples from HMVECs grown on collagen were compared to those from HMVECs grown on osteopontin. ESM-1 was determined to be up-regulated by more than 19-fold when grown on collagen. In an additional microarray experiment, gene expression from the RNA of HUVECs was compared to that from a pool of normal human tissues arising from six individuals (composed of tissues isolated from adipose, brain, breast, cervix, colon, heart, kidney, liver, skeletal muscle, lung, lymph node, pancreas, placenta, prostate, rectum, uterus, small intestine, spleen stomach, and testis). ESM-1 was found to be expressed approximately 11 times higher in the endothelial cells. Combining the results from these two experiments allows one to conclude that ESM-1 is expressed in endothelial cells and involved in the process of angiogenesis.

Based on this information, we decided to clone the full length open reading frame of ESM-1 cDNA by PCR from human dermal microvascular endothelial cells (HDMEC) RNA. Amplified product was then ligated into the pCRII-Topo vector and bacterial clones were isolated. The clones were then sequenced, confirming the predicted nucleic acid sequences (FIG. 1).

The cDNA isolated by PCR for the human ESM-1 gene was 2096 bp, which is good agreement with the predicted size by Northern analysis. The cDNA contains a 77 bp 5′ UTR, 555 bp coding region (FIG. 1A) and a 632 bp 3′ UTR. The putative initiation codon, aacATG, is good initiation context as judged by Kozak's rules (Kozak, 1999). A canonical polyadenylation signal, AATAAA, is present 20 bp upstream for the site of polyA addition. The 555 bp ORF codes a 184 amino acid protein with a molecular weight of 20096 daltons (FIG. 1A). Analysis of the deduced amino acid sequence by SignalP shows that the protein is predicted to have >99.9% probability to contain a cleaved 19 amino acid signal sequence at its N-terminus (FIG. 2, bottom). The predicted molecular of the protein lacking the signal sequence is 18123 daltons. The mature protein does not any putative sites for N-linked glycosylation but has a high cysteine content, containing 18 residues, which probably involved in many disulfide bonds, and is further evidence of a secretory protein.

Comparison to known functional domains and motifs revealed that amino acids 26-90 of the ESM-1 precursor protein contained significant homology to the insulin growth factor binding domain (FIG. 1B). This domain (pfam00219 also smart00121) is characteristic of a family protein homologs, which have a high affinity for insulin like growth factors. Members of this family include IGFBPs 1-6 (Shimasaki et al. 1991) and MAC25 (Murphy et al., 1993). The functional domains of the precursor human ESM-1 protein are further illustrated in FIG. 2 (top). The 19 amino acids of the premature protein contain a cleavable signal peptide with residues 26-90 containing the insulin growth factor binding domain (residues 7-71 in the mature protein). Comparison of the human ESM-1 protein against proteins from other species shows that there orthologs in rat and mouse (FIG. 3). This comparison reveals that the precursor ESM-1 protein is 184 amino acids in all three species with the human protein being 74.1% and 78.4% identical and 79.5% and 83.8% similar to the rat and mouse proteins, respectively.

Analysis of human genomic sequence shows that ESM-1 is located in 5p13 is transcribed from the upper DNA strand (FIG. 4). The primary transcript is processed as 3 exons of 378 bp, 150 bp and 1568 bp, respectively with each containing the canonical GT-AG splice junction. The first exon contains the 5′UTR and codes for the known functional domains, completely encompassing the signal sequence and the insulin growth factor binding domain. The second exon encodes an additional 50 amino acid and the third and final exon codes the remaining 35 amino acids and a large 3′ UTR (>1450 bp).

The fold change in the expression of ESM-1 mRNA in cancer was determined by microarray analysis (FIG. 5). Tumors from ten patients, each suffering from either breast, colon or kidney cancer or eleven patients having lung cancer were analyzed by comparing the ESM-1 mRNA expression with normal tissue from each tissue type. No breast cancer patients showed significant differential regulation of ESM-1. In contrast, three colon (FIG. 5, colon patient 2, 4 & 9) and kidney (kidney patient 2, 3 & 5) tumor patients and eight lung tumor patients (lung patients 4-11) showed >2-fold increases in the expression of the ESM-1 gene. The average increase in the expression of the three colon tumor patients was 2.28 fold and in the three kidney patients was 2.38 fold whereas the average increase in ESM-1 expression was greater than 3.10 fold in the eight lung tumor patients.

The microarray observations were then further validated in the cases of colon carcinoma by quantitative real time reverse transcription polymerase chain reaction (RT-RT-PCR). Nine of the ten colon tumors showed significant over-expression of human ESM-1, compared to the levels seen in a pool of normal colon tissue (FIG. 6). Levels of over-expression in these tumors ranged from 3-fold to over 50-fold.

RT-RTPCR was also performed on an array of colon tumor cell lines with the objective of determining the cells that expresses human ESM-1 (FIG. 7). When compared to the cell line DLD-1, which had the lowest levels of RNA, the colon cell lines showed a range of variation of expression. The SW480 and HCT116 lines had between 11 and 26-fold higher expression while the VM46 cells show nearly one thousand times higher levels of ESM-1. This data will be useful in the establishment of animal models for modulating the gene.

In order to determine if ESM-1 is expressed in the endothelium of tumors, we utilized a transgenic mouse that contains the promoter sequence from Tie2 combined with the green fluorescent protein (GFP) gene (Schlaeger et al., 1997), which had been backcrossed into the C57/B6 strain background. The reporter gene in this mouse is expressed exclusively and uniformly throughout all vascular endothelial cells. Mice containing this transgene were injected with either lewis lung tumor cell line or melanoma B16 line and the tumor is allowed several weeks to become established. Tumors are removed and endothelial cells are then sorted by FACS from the tumors and RNA is isolated from both the endothelial cells and from the remaining tumor cell population which has been endothelial cell depleted (reference or materials and methods). Quantitative real time RT-PCR shows the ESM-1 gene to be expressed at considerably higher levels in the endothelial cell RNA than the corresponding tumor cell population, which has been depleted of endothelial cell RNA (FIG. 8). Endothelial cells from the Lewis lung tumor show about 68-fold higher expression of ESM-1 when compared to the tumor epithelial cells while B16 derived endothelial cells demonstrate greater than 45-fold over-expression compared to B16 tumor cell population depleted of endothelial cells.

In order to assess the potential of ESM-1 to induce or accentuate tumor formation, the gene was shuttled into a CMV promoter-driven expression construct and transfected into NIH3T3 cells. Stable pools of NIH3T3 cells were assayed for ESM-1 transgene expression (FIG. 9A). Cells were then injected subcutaneous into female CD-1 nu/nu mice and evaluated over time for growth. The NIH3T3 cells that over-express the ESM-1 gene showed significant enhancement of growth when compared to the same cells transfected with a vector control (FIG. 9B), showing ESM-1 can act to enhance the growth of fibroblasts.

In situ hybridization analysis was performed on over 300 tumor samples of various types, including colon, renal, breast, ovary, prostate, and lung carcinomas, as well as non-tumor tissues (FIG. 10). ESM-1 was found to be expressed in 28.1% of renal cell carcinomas and 9.6% of breast carcinomas. Little to no staining was seen for the normal tissues. Serial sections of the tumor samples were immuno histochemically stained with human Factor VIII antibody (DAKO antibody #M0616) in order to identify the blood vessels in each section. Tumors were then grouped into those showing ESM-1 expression and those without ESM-1 expression. There is a statistically significant correlation between those tumors showing ESM-1 expression and blood vessel density as determined by Factor VIII (FIG. 11). This implies that ESM-1 may play a role in the process of angiogenesis.

One embodiment of the present invention is nucleic acid sequences that encode an ESM-1 polypeptide. Preferably the nucleic acid is selected from the group consisting of:

a nucleic acid sequence capable of hybridizing under stringent conditions, or which would be capable of hybridizing under said conditions but for the degeneracy of the genetic code, to the DNA sequence of SEQ ID NO:1;

a nucleic acid sequence having at least about 80% homology to the DNA sequence of SEQ ID NO:1; and

a complement sequence of SEQ ID NO:1.

Another embodiment of the present invention is an ESM-1 amino acid sequence of SEQ ID NO:2 and the mature secreted form of ESM-1, amino acids 20-184 of SEQ ID NO:2 (SEQ ID NO:19). The amino acid sequence may be selected from the group consisting of:

an amino acid sequence having at least about 70% homology to the amino acid sequence of SEQ ID NO:2, or SEQ ID NO:19;

a substitution, deletion or insertion variant of the amino acid sequence of SEQ ID NO:2, or SEQ ID NO:19; and

an allelic variant of SEQ ID NO:2, or SEQ ID NO:19.

Sequence Variants

DNA encoding amino acid sequence variants of ESM-1 can be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of ESM-1. These techniques may utilize ESM-1 nucleic acid (DNA or RNA), or nucleic acid complementary to ESM-1 nucleic acid.

Oligonucleotide-mediated mutagenesis is a preferred method for preparing substitution, deletion, and insertion variants of ESM-1 DNA. This technique is well known in the art, for example as described by Adelman et al., DNA, 2: 183 (1983). Briefly, ESM-1 DNA is altered by hybridizing an oligonucleotide encoding the desired mutation to a DNA template, where the template is the single-stranded form of a plasmid or bacteriophage containing the unaltered or native DNA sequence of ESM-1. After hybridization, a DNA polymerase is used to synthesize an entire second complementary strand of the template that will thus incorporate the oligonucleotide primer, and will code for the selected alteration in ESM-1 DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765, 1978).

Single-stranded DNA template may also be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase 1, is then added to synthesize the complementary strand of the template using the oligonucleotide as a primer for synthesis. A heteroduplex molecule is thus formed such that one strand of DNA encodes the mutated form of ESM-1, and the other strand (the original template) encodes the native, unaltered sequence of ESM-1. This heteroduplex molecule is then transformed into a suitable host cell, usually a prokaryote such as E. coli JM101. The cells are plated onto agarose plates, and screened using the oligonucleotide primer radiolabeled with ³²-phosphate to identify the bacterial colonies that contain the mutated DNA. The mutated region is then removed and placed in an appropriate vector for protein production, generally an expression vector of the type typically employed for transformation of an appropriate host.

The method described immediately above may be modified such that a homoduplex molecule is created wherein both strands of the plasmid contain the mutation(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thio-deoxyribocytosine called dCTP-(α³⁵S) (which can be obtained from Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(α³⁵S) instead of dCTP, which serves to protect it from restriction endonuclease digestion. After the template strand of the double-stranded heteroduplex is nicked with an appropriate restriction enzyme, the template strand can be digested with Exo III nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101, as described above.

DNA encoding ESM-1 mutants with more than one amino acids to be substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions.

The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

PCR mutagenesis is also suitable for making amino acid variants of ESM-1. While the following discussion refers to DNA, it is understood that the technique also finds application with RNA. The PCR technique generally refers to the following procedure (see Erlich, supra, the chapter by R. Higuchi, p. 61-70): When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers is designed to overlap the position of the mutation and to contain the mutation; the sequence of the other primer must be identical to a stretch of sequence of the opposite strand of the plasmid, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR amplification using a primer pair like the one just described results in a population of DNA fragments that differ at the position of the mutation specified by the primer, and possibly at other positions, as template copying is somewhat error-prone.

If the ratio of template to product material is extremely low, the vast majority of product DNA fragments incorporate the desired mutation(s). This product material is used to replace the corresponding region in the plasmid that served as PCR template using standard DNA technology. Mutations at separate positions can be introduced simultaneously by either using a mutant second primer, or performing a second PCR with different mutant primers and ligating the two resulting PCR fragments simultaneously to the vector fragment in a three (or more)-part ligation.

Another method for preparing variants, cassette mutagenesis, is based on the technique described by Wells et al. (Gene, 34: 315>1985). The starting material is the plasmid (or other vector) comprising ESM-1 DNA to be mutated. The codon(s) in ESM-1 DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in ESM-1 DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ends that are compatible with the ends of the linearized plasmid, such that it can be directly ligated to the plasmid. This plasmid now contains the mutated ESM-1 DNA sequence.

Covalent Modification of Proteins

Covalent modifications of a protein or antibodies of the present invention are included within the scope of this invention. One type of covalent modification includes reacting targeted amino acid residues of a polypeptide with an organic derivatizing agent that is capable of reacting with selected side chains or the N- or C-terminal residues of the a protein of the present invention. Derivatization with bifunctional agents is useful, for instance, for crosslinking protein to a water-insoluble support matrix or surface for use in the method for purifying antibodies, and vice-versa. Commonly used crosslinking agents include e. g. 1,1-bis(diazoacetyl)-2-phenylethane glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(pazidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the —amino groups of lysine, arginine, and histidine side chains (See T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the polypeptide included within the scope of this invention comprises altering the native glycosylation pattern of the polypeptide. “Altering the native glycosylation pattern” is intended for purposes herein to mean deleting one or more carbohydrate moieties found in the native sequence (either by removing the underlying glycosylation site or by deleting the glycosylation by chemical and/or enzymatic means), and/or adding one or more glycosylation sites that are not present in the native sequence. In addition, the phrase includes qualitative changes in the glycosylation of the native proteins, involving a change in the nature and proportions of the various carbohydrate moieties present. Addition of glycosylation sites to the polypeptide can be accomplished by altering the amino acid sequence. The alteration can be made, for example, by the addition of, or substitution by, one or more serine or threonine residues to the native sequence (for Olinked glycosylation sites). The amino acid sequence can optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide can be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys, 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a protein or antibody of the present invention comprises linking the polypeptide or antibody to one of a variety of non-proteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

Functional groups capable of reacting with either the amino terminal α-amino group or ε-amino groups of lysines found on the ESM-1, agonist, antagonist, or antibody include: carbonates such as the p-nitrophenyl, or succinimidyl; carbonyl imidazole; azlactones; cyclic imide thiones; isocyanates or isothiocyanates; tresyl chloride (EP 714 402, EP 439 508); and aldehydes. Functional groups capable of reacting with carboxylic acid groups, reactive carbonyl groups and oxidized carbohydrate moieties on ESM-1, agonist, antagonist, or antibody include; primary amines; and hydrazine and hydrazide functional groups such as the acyl hydrazides, carbazates, semicarbamates, thiocarbazates, etc. Mercapto groups, if available on the ESM-1, agonist, antagonist, or antibody, can also be used as attachment sites for suitably activated polymers with reactive groups such as thiols; maleimides, sulfones, and phenyl glyoxals; see, for example, U.S. Pat. No. 5,093,531, the disclosure of which is hereby incorporated by reference. Other nucleophiles capable of reacting with an electrophilic center include, but are not limited to, for example, hydroxyl, amino, carboxyl, thiol, active methylene and the like.

In one preferred embodiment of the invention secondary amine or amide linkages are formed using the ESM-1, agonist, antagonist, or antibody N-terminal amino groups or ε-amino groups of lysine and the activated PEG. In another preferred aspect of the invention, a secondary amine linkage is formed between the N-terminal primary amino group of ESM-1, agonist, antagonist, or antibody and single or branched chain PEG aldehyde by reduction with a suitable reducing agent such as NaCNBH₃, NaBH₃, Pyridine Borane etc. as described in Chamow et al., Bioconjugate Chem. 5: 133-140 (1994) and U.S. Pat. No. 5,824,784.

In another preferred embodiment of the invention, polymers activated with amide-forming linkers such as succinimidyl esters, cyclic imide thiones, or the like are used to effect the linkage between the ESM-1, agonist, antagonist, or antibody and polymer, see for example, U.S. Pat. No. 5,349,001; U.S. Pat. No. 5,405,877; and Greenwald, et al., Crit. Rev. Ther. Drug Carrier Syst. 17:101-161, 2000, which are incorporated herein by reference. One preferred activated poly(ethylene glycol), which may be bound to the free amino groups of ESM-1, agonist, antagonist, or antibody includes single or branched chain N-hydroxysuccinylimide poly(ethylene glycol) may be prepared by activating succinic acid esters of poly(ethylene glycol) with N-hydroxysuccinylimide.

Other preferred embodiments of the invention include using other activated polymers to form covalent linkages of the polymer with the ESM-1, agonist, antagonist, or antibody via ε-amino or other groups. For example, isocyanate or isothiocyanate forms of terminally activated polymers can be used to form urea or thiourea-based linkages with the lysine amino groups.

In another preferred aspect of the invention, carbamate (urethane) linkages are formed with protein amino groups as described in U.S. Pat. Nos. 5,122,614, 5,324,844, and 5,612,640, which are hereby incorporated by reference. Examples include N-succinimidyl carbonate, para-nitrophenyl carbonate, and carbonyl imidazole activated polymers. In another preferred embodiment of this invention, a benzotriazole carbonate derivative of PEG is linked to amino groups on ESM-1, agonist, antagonist, or antibody.

Insertion of DNA into a Cloning Vehicle

The cDNA or genomic DNA encoding native or variant ESM-1 is inserted into a replicable vector for further cloning (amplification of the DNA) or for expression. Many vectors are available, and selection of the appropriate vector will depend on 1) whether it is to be used for DNA amplification or for DNA expression, 2) the size of the DNA to be inserted into the vector, and 3) the host cell to be transformed with the vector. Each vector contains various components depending on its function (amplification of DNA or expression of DNA) and the host cell for which it is compatible. The vector components generally include, but are not limited to, one or more of the following: a signal sequence, an origin of replication, one or more marker genes, an enhancer element, a promoter, and a transcription termination sequence.

Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Most expression vectors are “shuttle” vectors, i.e. they are capable of replication in at least one class of organisms but can be transfected into another organism for expression. For example, a vector is cloned in E. Coli and then the same vector is transfected into yeast or mammalian cells for expression even though it is not capable of replicating independently of the host cell chromosome.

DNA may also be amplified by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of the ESM-1 DNA. However, the recovery of genomic DNA encoding ESM-1 is more complex than that of an exogenously replicated vector because restriction enzyme digestion is required to excise ESM-1 DNA.

Selection Gene Component

Expression and cloning vectors should contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g. ampicillin, neomycin, methotrexate, or tetracycline, (b) complement auxotrophic deficiencies, or (c) supply critical nutrients not available from complex media, e.g. the gene encoding D-alanine racemase for Bacilli.

One example of a selection scheme utilizes a drug to arrest growth of a host cell. Those cells that are successfully transformed with a heterologous gene express a protein conferring drug resistance and thus survive the selection regimen. Examples of such dominant selection use the drugs neomycin (Southern et al., J. Molec. Appl. Appl. Genet., 1: 327>1982), mycophenolic acid (Mulligan et al., Science, 209: 1422>1980) or hygromycin (Sugden et al., Mol. Cell. Biol., 5: 410-413>1985). The three examples given above employ bacterial genes under eukaryotic control to convey resistance to the appropriate drug G418 or neomycin (geneticin), xgpt (mycophenolic acid), or hygromycin, respectively.

Another example of suitable selectable markers for mammalian cells are those that enable the identification of cells competent to take up ESM-1 nucleic acid, such as dihydrofolate reductase (DHFR) or thymidine kinase. The mammalian cell transformants are placed under selection pressure, which only the transformants are uniquely adapted to survive by virtue of having taken up the marker. Selection pressure is imposed by culturing the transformants under conditions in which the concentration of selection agent in the medium is successively changed, thereby leading to amplification of both the selection gene and the DNA that encodes ESM-1. Amplification is the process by which genes in greater demand for the production of a protein critical for growth are reiterated in tandem within the chromosomes of successive generations of recombinant cells. Increased quantities of ESM-1 are synthesized from the amplified DNA.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub and Chasin, Proc. Natl. Acad. Sci. USA, 77: 4216>1980. The transformed cells are then exposed to increased levels of methotrexate. This leads to the synthesis of multiple copies of the DHFR gene, and, concomitantly, multiple copies of other DNA comprising the expression vectors, such as the DNA encoding the PF4A receptor. This amplification technique can be used with any otherwise suitable host, e.g., ATCC No. CCL61 CHO-K1, notwithstanding the presence of endogenous DHFR if, for example, a mutant DHFR gene that is highly resistant to Mtx is employed (EP 117,060). Alternatively, host cells (particularly wild-type hosts that contain endogenous DHFR) transformed or co-transformed with DNA sequences encoding the PF4A receptor, wild-type DHFR protein, and another selectable marker such as aminoglycoside 3′ phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282: 39>1979; Kingsman et al., Gene, 7: 141>1979; or Tschemper et al., Gene, 10: 157>1980). The trp1 gene provides a selection marker for a mutant strain of yeast lacking the ability to grow in tryptophan, for example, ATCC No. 44076 or PEP4-1 (Jones, Genetics, 85: 12>1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

Promoter Component

Expression and cloning vectors usually contain a promoter that is recognized by the host organism and is operably linked to the ESM-1 nucleic acid. Promoters are untranslated sequences located upstream (5′) to the start codon of a structural gene (generally within about 100 to 1000 bp) that control the transcription and translation of a particular nucleic acid sequence, such as ESM-1, to which they are operably linked. Such promoters typically fall into two classes, inducible and constitutive. Inducible promoters are promoters that initiate increased levels of transcription from DNA under their control in response to some change in culture conditions, e.g. the presence or absence of a nutrient or a change in temperature. At this time a large number of promoters recognized by a variety of potential host cells are well known. These promoters are operably linked to DNA encoding ESM-1 by removing the promoter from the source DNA by restriction enzyme digestion and inserting the isolated promoter sequence into the vector. Both the native ESM-1 promoter sequence and many heterologous promoters may be used to direct amplification and/or expression of ESM-1 DNA. However, heterologous promoters are preferred, as they generally permit greater transcription and higher yields of expressed ESM-1 as compared to the native ESM-1 promoter.

Promoters suitable for use with prokaryotic hosts include the β-lactamase and lactose promoter systems (Chang et al., Nature, 275: 615>1978; and Goeddel et al., Nature, 281: 544>1979), alkaline phosphatase, a tryptophan (trp) promoter system (Goeddel, Nucleic Acids Res., 8: 4057>1980 and EP 36,776) and hybrid promoters such as the tac promoter (deBoer et al., Proc. Natl. Acad. Sci. USA, 20: 21-25>1983). However, other known bacterial promoters are suitable. Their nucleotide sequences have been published, thereby enabling a skilled worker operably to ligate them to DNA encoding ESM-1 (Siebenlist et al., Cell, 20: 269>1980) using linkers or adaptors to supply any required restriction sites. Promoters for use in bacterial systems also generally will contain a Shine-Dalgarno (S.D.) sequence operably linked to the DNA encoding ESM-1.

Suitable promoting sequences for use with yeast hosts include the promoters for 3-phosphoglycerate kinase (Hitzeman et al., J. Biol. Chem., 255: 2073>1980) or other glycolytic enzymes (Hess et al., J. Adv. Enzyme Reg., 7: 149>1968; and Holland, Biochemistry, 17: 4900>1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in Hitzeman et al., EP 73,657A. Yeast enhancers also are advantageously used with yeast promoters.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X may be any nucleotide. At the 3′end of most eukaryotic genes is an AATAAA sequence that may be the signal for addition of the poly A tail to the 3′end of the coding sequence. All of these sequences are suitably inserted into mammalian expression vectors.

ESM-1 transcription from vectors in mammalian host cells is controlled by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g. the actin promoter or an immunoglobulin promoter, from heat-shock promoters, and from the promoter normally associated with ESM-1 sequence, provided such promoters are compatible with the host cell systems.

The early and late promoters of the SV40 virus are conveniently obtained as an SV40 restriction fragment that also contains the SV40 viral origin of replication. Fiers et al., Nature, 273:113 (1978); Mulligan and Berg, Science, 209: 1422-1427 (1980); Pavlakis et al., Proc. Natl. Acad. Sci. USA, 78: 7398-7402 (1981). The immediate early promoter of the human cytomegalovirus is conveniently obtained as a HindIII E restriction fragment. Greenaway et al., Gene, 18: 355-360 (1982). A system for expressing DNA in mammalian hosts using the bovine papilloma virus as a vector is disclosed in U.S. Pat. No. 4,419,446. A modification of this system is described in U.S. Pat. No. 4,601,978. See also Gray et al., Nature, 295: 503-508 (1982) on expressing cDNA encoding immune interferon in monkey cells; Reyes et al., Nature, 297: 598-601 (1982) on expression of human β-interferon cDNA in mouse cells under the control of a thymidine kinase promoter from herpes simplex virus, Canaani and Berg, Proc. Natl. Acad. Sci. USA, 79: 5166-5170 (1982) on expression of the human interferon β1 gene in cultured mouse and rabbit cells, and Gorman et al., Proc. Natl. Acad. Sci. USA, 79: 6777-6781 (1982) on expression of bacterial CAT sequences in CV-1 monkey kidney cells, chicken embryo fibroblasts, Chinese hamster ovary cells, HeLa cells, and mouse NIH-3T3 cells using the Rous sarcoma virus long terminal repeat as a promoter.

Enhancer Element Component

Transcription of a DNA encoding ESM-1 of this invention by higher eukaryotes is often increased by inserting an enhancer sequence into the vector. Enhancers are cis-acting elements of DNA, usually about from 10-300 bp that act on a promoter to increase its transcription. Enhancers are relatively orientation and position independent having been found 5′ (Laimins et al., Proc. Natl. Acad. Sci. USA, 78: 993>1981) and 3′ (Lusky et al., Mol. Cell Bio., 3: 1108>1983) to the transcription unit, within an intron (Baneiji et al., Cell, 33: 729>1983) as well as within the coding sequence itself (Osborne et al., Mol. Cell Bio., 4: 1293>1984). Many enhancer sequences are now known from mammalian genes (globin, elastase, albumin, α-fetoprotein and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297: 17-18 (1982) on enhancing elements for activation of eukaryotic promoters. The enhancer may be spliced into the vector at a position 5′ or 3′ to the ESM-1 DNA, but is preferably located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′untranslated regions of eukaryotic or viral DNAs or cDNAs. These regions contain nucleotide segments transcribed as polyadenylated fragments in the untranslated portion of the mRNA encoding ESM-1. The 3′untranslated regions also include transcription termination sites.

Construction of suitable vectors containing one or more of the above listed components the desired coding and control sequences employs standard ligation techniques. Isolated plasmids or DNA fragments are cleaved, tailored, and religated in the form desired to generate the plasmids required.

For analysis to confirm correct sequences in plasmids constructed, the ligation mixtures are used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced by the method of Messing et al., Nucleic Acids Res., 9: 309 (1981) or by the method of Maxam et al., Methods in Enzymology, 65: 499 (1980).

Particularly useful in the practice of this invention are expression vectors that provide for the transient expression in mammalian cells of DNA encoding ESM-1. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of ESM-1 that have ESM-1-like activity.

Other methods, vectors, and host cells suitable for adaptation to the synthesis of the ESM-1 in recombinant vertebrate cell culture are described in Gething et al., Nature, 293: 620-625>1981; Mantei et al., Nature, 281: 40-46>1979; Levinson et al.; EP 117,060; and EP 117,058. A particularly useful plasmid for mammalian cell culture expression of the PF4A receptor is pRK5 (EP pub. no. 307,247) or pSVI6B (U.S. Ser. No. 07/441,574 filed Nov. 22, 1989, the disclosure of which is incorporated herein by reference).

Selection and Transformation of Host Cells

Suitable host cells for cloning or expressing the vectors herein are the prokaryote, yeast, or higher eukaryotic cells described above. Suitable prokaryotes include eubacteria, such as Gram-negative or Gram-positive organisms, for example, E. coli, Bacilli such as B. subtilis, Pseudomonas species such as P. aerupinosa, Salmonella typhimurium, or Serratia marcescens. One preferred E. coli cloning host is E. coli 294 (ATCC 31,446), although other strains such as E. coli B, E. coli chi-1776 (ATCC 31,537), and E. coli W3110 (ATCC 27,325) are suitable. These examples are illustrative rather than limiting. Preferably the host cell should secrete minimal amounts of proteolytic enzymes. Alternatively, in vitro methods of cloning, e.g. PCR or other nucleic acid polymerase reactions are suitable.

In addition to prokaryotes, eukaryotic microbes such as filamentous fungi or yeast are suitable hosts for vectors containing ESM-1 DNA. Saccharomyces cerevisiae, or common baker's yeast, is the most commonly used among lower eukaryotic host microorganisms. However, a number of other genera, species, and strains are commonly available and useful herein, such as S. pombe>Beach and Nurse, Nature, 290: 140 (1981), Kluyveromyces lactis>Louvencourt et al., J. Bacteriol., 737 (1983), yarrowia>EP 402,226, Pichia pastoris>EP 183,070, Trichoderma reesia>EP 244,234, Neurospora crassa>Case et al., Proc. Natl. Acad. Sci. USA, 76: 5259-5263 (1979), and Aspergillus hosts such as A. nidulans>Ballance at al., Biochem. Biophys. Res. Commun., 112: 284-289 (1983); Tilburn et al., Gene, 26: 205-221 (1983); Yelton et al., Proc. Natl. Acad. Sci. USA, 81: 1470-1474 (1984) and A. niger>Kelly and Hynes, EMBO J., 4: 475-479 (1985).

Suitable host cells for the expression of glycosylated ESM-1 polypeptide are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori host cells have been identified. See, e.g., Luckow et al., Bio Technology, 6: 47-55 (1988); Miller et al., in Genetic Engineering, Setlow, J. K. et al., eds., Vol. 8 (Plenum Publishing, 1986), pp. 277-279; and Maeda et al., Nature, 315: 592-594 (1985). A variety of such viral strains are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can be utilized as hosts. Typically, plant cells are transfected by incubation with certain strains of the bacterium Agrobacterium tumefaciens, which has been previously manipulated to contain ESM-1 DNA. During incubation of the plant cell culture with A. tumefaciens, the DNA encoding ESM-1 is transferred to the plant cell host such that it is transfected, and will, under appropriate conditions, express ESM-1 DNA. In addition, regulatory and signal sequences compatible with plant cells are available, such as the nopaline synthase promoter and polyadenylation signal sequences. Depicker et al., J. Mol. Appl. Gene, 1: 561 (1982). In addition, DNA segments isolated from the upstream region of the T-DNA 780 gene are capable of activating or increasing transcription levels of plant-expressible genes in recombinant DNA-containing plant tissue. See EP 321,196 published Jun. 21, 1989.

However, interest has been greatest in vertebrate cells, and propagation of vertebrate cells in culture (tissue culture) has become a routine procedure in recent years (Tissue Culture, Academic Press, Kruse and Patterson, editors (1973)). Examples of useful mammalian host cell lines are monkey kidney CV1 line transformed by SV40 (COS-7, ATCC CRL 1651); human embryonic kidney line (293 or 293 cells subcloned for growth in suspension culture, (Graham et al., J. Gen Virol., 36: 59 (1977)); baby hamster kidney cells (BHK, ATCC CCL 10); Chinese hamster ovary cells/-DHFR (CHO, Urlaub and Chasin, Proc. Natl. Acad. Sci. USA, 77: 4216>1980); mouse sertoli cells (TM4, Mather, Biol. Reprod., 23: 243-251>1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383: 44-68>1982); MRC 5 cells; FS4 cells; and a human hepatoma cell line (Hep G2). Preferred host cells are human embryonic kidney 293 and Chinese hamster ovary cells.

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al. supra, is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859 published Jun. 29, 1989. For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216 issued Aug. 16, 1983. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells such as by nuclear injection, electroporation, or by protoplast fusion may also be used.

Culturing the Host Cells

Prokaryotic cells used to produce ESM-1 polypeptide of this invention are cultured in suitable media as described generally in Sambrook et al.

The mammalian host cells used to produce ESM-1 of this invention may be cultured in a variety of media. Commercially available media such as Ham's F10 (Sigma), Minimal Essential Medium (>MEM, Sigma), RPMI-1640 (Sigma), and Dulbecco's Modified Eagle's Medium (>DMEM, Sigma) are suitable for culturing the host cells. In addition, any of the media described in Ham and Wallace, Meth. Enz., 58: 44 (1979), Barnes and Sato, Anal. Biochem., 102: 255 (1980), U.S. Pat. Nos. 4,767,704; 4,657,866; 4,927,762; or 4,560,655; WO 90/03430; WO 87/00195; U.S. Pat. No. Re. 30,985; or copending U.S. Ser. No. 07/592,107 or 07/592,141, both filed in Oct. 3, 1990, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in vitro culture as well as cells that are within a host animal.

It is further envisioned that ESM-1 of this invention may be produced by homologous recombination, or with recombinant production methods utilizing control elements introduced into cells already containing DNA encoding ESM-1. For example, a powerful promoter/enhancer element, a suppressor, or an exogenous transcription modulator element is inserted in the genome of the intended host cell in proximity and orientation sufficient to influence the transcription of DNA encoding the desired ESM-1. The control element does not encode the ESM-1 of this invention, but the DNA is present in the host cell genome. One next screens for cells making ESM-1 of this invention, or increased or decreased levels of expression, as desired.

Therapeutic Compositions and Administration of ESM-1

Therapeutic formulations of ESM-1 are prepared for storage by mixing ESM-1 having the desired degree of purity with optional physiologically acceptable carriers, excipients, or stabilizers (Remington's Pharmaceutical Sciences, supra.), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The compositions useful in the treatment of cardiovascular, endothelial, oncological, and angiogenic disorders include, without limitation, antibodies, small organic and inorganic molecules, peptides, phosphopeptides, antisense and ribozyme molecules, triple-helix molecules, etc., that inhibit the expression and/or activity of the target gene product.

While it is possible for an active ingredient to be administered alone as the raw chemical, it is preferable to present it as a pharmaceutical formulation. The present invention comprises a pharmaceutical composition comprising a therapeutically effective amount of a compound of the present invention in association with at least one pharmaceutically acceptable carrier, adjuvant, or diluent. The present invention also comprises a method of treating inflammation or inflammation associated disorders in a subject, the method comprising administering to the subject having such inflammation or disorders a therapeutically effective amount of a compound of the present invention. Also included in the family of compounds of the present invention are the pharmaceutically acceptable salts thereof. The term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, phydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, cyclohexylaminosulfonic, algenic, β-hydroxybutyric, salicyclic, galactaric and galacturonic acid. Suitable pharmaceutically acceptable base addition salts of compounds of the present invention include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methyl-glucamine) and procaine. All of these salts may be prepared by conventional means from the corresponding compound of the present invention by reacting, for example, the appropriate acid or base with the compound of the present invention.

Also embraced within this invention are pharmaceutical compositions comprising one or more compounds of the present invention in association with one or more non-toxic, pharmaceutically acceptable carriers and/or diluents and/or adjuvants and/or excipient (collectively referred to herein as “carrier” materials) and, if desired, other active ingredients. Accordingly, the compounds of the present invention may be used in the manufacture of a medicament. Pharmaceutical compositions of the compounds of the present invention prepared as herein before described may be formulated as solutions or lyophilized powders for parenteral administration. Powders may be reconstituted by addition of a suitable diluent or other pharmaceutically acceptable carrier prior to use. The liquid formulation may be a buffered, isotonic aqueous solution. The compounds of the present invention may be administered by any suitable route, preferably in the form of a pharmaceutical composition adapted to such a route, and in a dose effective for the treatment intended. The compounds and composition may, for example, be administered intravascularly, intraperitoneally, intravenously, subcutaneously, intramuscularly, intramedullary, orally, or topically. For oral administration, the pharmaceutical composition may be in the form of, for example, a tablet, capsule, suspension, or liquid. The active ingredient may also be administered by injection as a composition wherein, for example, normal isotonic saline solution, standard 5% dextrose in water or buffered sodium or ammonium acetate solution may be used as a suitable carrier. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, or sodium citrate. The pharmaceutical composition is preferably made in the form of a dosage unit containing a particular amount of the active ingredient. Examples of such dosage units are tablets or capsules. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredient in the range of about 0.1 to 2000 mg, preferably in the range of about 0.5 to 500 mg and most preferably between about 1 and 100 mg. A daily dose of about 0.01 to 100 mg/kg bodyweight, preferably between about 0.1 and about 50 mg/kg body weight and most preferably between about 1 to 20 mg/kg bodyweight, may be appropriate. The daily dose can be administered in one to four doses per day. For therapeutic purposes, the compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled release formulation as may be provided in a dispersion of active compound in a sustained release material such as glyceryl monostearate, glyceryl distearate, hydroxypropylmethyl cellulose alone or with a wax. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion, or an aqueous or non-aqueous suspension. Such a liquid formulation may be administered orally or filled into a soft gelatin capsule. For rectal administration, the compounds of the present invention may also be combined with excipients such as cocoa butter, glycerin, gelatin, or polyethylene glycols and molded into a suppository. The methods of the present invention include topical administration of the compounds of the present invention. By topical administration is meant non-systemic administration, including the application of a compound of the invention externally to the epidermis, to the buccal cavity and instillation of such a compound into the ear, eye, and nose, wherein the compound does not significantly enter the blood stream. By systemic administration is meant oral, intravenous, intraperitoneal, and intramuscular administration. The amount of a compound of the present invention (hereinafter referred to as the active ingredient) required for therapeutic or prophylactic effect upon topical administration will, of course, vary with the compound chosen, the nature and severity of the condition being treated and the animal undergoing treatment, and is ultimately at the discretion of the physician.

The topical formulations of the present invention, both for veterinary and for human medical use, comprise an active ingredient together with one or more acceptable carriers therefore, and optionally any other therapeutic ingredients. The carrier must be “acceptable” in the sense of being compatible with the other ingredients of the formulation and not deleterious to the recipient thereof. Formulations suitable for topical administration include liquid or semi- liquid preparations suitable for penetration through the skin to the site of where treatment is required such as: liniments, lotions, creams, ointments or pastes, and drops suitable for administration to the eye, ear or nose. The active ingredient may comprise, for topical administration, from 0.01 to 5.0 wt % of the formulation.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container, which is then sealed and sterilized by autoclaving, or maintaining at 90-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.00217c), benzalkonium chloride (0.01%) and chlorhexidine acetate (0.01%). Suitable solvents for the preparation of an oily solution include glycerol, diluted alcohol, and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or oil such as castor oil or arachis oil. Creams, ointments, or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The formulation may incorporate any suitable surface-active agent such as an anionic, cationic, or non-ionic surface-active agent such as sorbitan esters or polyoxyethylene derivatives thereof. Suspending agents such as natural gums, cellulose derivatives or inorganic materials such as silicaceous silicas, and other ingredients such as lanolin may also be included. Other adjuvants and modes of administration are well and widely known in the pharmaceutical art. Although this invention has been described with respect to specific embodiments, the details of these embodiments are not to be construed as limitations.

ESM-1 or fragments to be used for in vivo administration must be sterile. This is readily accomplished by filtration through sterile filtration membranes, prior to or following lyophilization and reconstitution.

Therapeutic ESM-1 compositions generally are placed into a container having a sterile access port, for example, an intravenous solution bag, or vial having a stopper pierceable by a hypodermic injection needle.

The route of ESM-1 or ESM-1 antibody administration is in accord with known methods, e.g. injection or infusion by intravenous, intraperitoneal, intracerebral, intramuscular, intraocular, intraarterial, or intralesional routes, or by sustained release systems as noted below. ESM-1 or fragment is administered continuously by infusion or by bolus injection. ESM-1 antibody is administered in the same fashion, or by administration into the blood stream or lymph.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al., Biopolymers, 22: 547-556>1983), poly (2-hydroxyethylmethacrylate) (Langer et al., J. Biomed. Mater. Res., 15: 167-277>1981, and Langer, Chem. Tech., 12: 98-105>1982), ethylene vinyl acetate (Langer et al., supra) or poly-D-(−)-3-hydroxybutyric acid (EP 133,988). Sustained-release ESM-1 compositions also include liposomally entrapped ESM-1. Liposomes containing ESM-1 are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102 324. Ordinarily the liposomes are of the small (about 200-800 Angstroms) unilamelar type in which the lipid content is greater than about 30% cholesterol, the selected proportion being adjusted for the optimal ESM-1 therapy.

An effective amount of ESM-1 to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer the ESM-1 or fragment until a dosage is reached that achieves the desired effect. The progress of this therapy is easily monitored by conventional assays.

Analytical methods for ESM-1 or its antibodies all use one or more of the following reagents: labeled analyte analogue, immobilized analyte analogue, labeled binding partner, immobilized binding partner and steric conjugates. The labeled reagents also are known as “tracers.” The label used (and this is also useful to label ESM-1 nucleic acid for use as a probe) is any detectable functionality that does not interfere with the binding of analyte and its binding partner. Numerous labels are known for use in immunoassay, examples including moieties that may be detected directly, such as fluorochrome, chemiluminescent, and radioactive labels, as well as moieties, such as enzymes, that must be reacted or derivatized to be detected. Examples of such labels include the radioisotopes ³²p, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al., Nature, 144: 945 (1962); David et al., Biochemistry, 13: 1014-1021 (1974); Pain et al., J. Immunol. Methods, 40: 219-230 (1981); and Nygren, J. Histochem. Cytochem., 30: 407-412 (1982). Preferred labels herein are enzymes such as horseradish peroxidase and alkaline phosphatase. The conjugation of such label, including the enzymes, to the antibody is a standard manipulative procedure for one of ordinary skill in immunoassay techniques. See, for example, O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166. Such bonding methods are suitable for use with ESM-1 or its antibodies, all of which are proteinaceous.

Immobilization of reagents is required for certain assay methods. Immobilization entails separating the binding partner from any analyte that remains free in solution. This conventionally is accomplished by either insolubilizing the binding partner or analyte analogue before the assay procedure, as by adsorption to a water-insoluble matrix or surface (Bennich et al., U.S. Pat. No. 3,720,760), by covalent coupling (for example, using glutaraldehyde cross-linking), or by insolubilizing the partner or analogue afterward, e.g., by immunoprecipitation.

Other assay methods, known as competitive or sandwich assays, are well established and widely used in the commercial diagnostics industry.

Competitive assays rely on the ability of a tracer analogue to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results to quantitatively determine the amount of analyte present in the test sample. These assays are called ELISA systems when enzymes are used as the detectable markers.

Another species of competitive assay, called a “homogeneous” assay, does not require a phase separation. Here, a conjugate of an enzyme with the analyte is prepared and used such that when anti-analyte binds to the analyte the presence of the anti-analyte modifies the enzyme activity. In this case, ESM-1 or its immunologically active fragments are conjugated with a bifunctional organic bridge to an enzyme such as peroxidase. Conjugates are selected for use with anti-ESM-1 so that binding of the anti-ESM-1 inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small analyte so that antibody to hapten substantially is unable to bind the conjugate at the same time as anti-analyte. Under this assay procedure the analyte present in the test sample will bind anti-analyte, thereby allowing anti-hapten to bind the conjugate, resulting in a change in the character of the conjugate hapten, e.g., a change in fluorescence when the hapten is a fluorophore.

Sandwich assays particularly are useful for the determination of ESM-1 or ESM-1 antibodies. In sequential sandwich assays an immobilized binding partner is used to adsorb test sample analyte, the test sample is removed as by washing, the bound analyte is used to adsorb labeled binding partner, and bound material is then separated from residual tracer. The amount of bound tracer is directly proportional to test sample analyte. In “simultaneous” sandwich assays the test sample is not separated before adding the labeled binding partner. A sequential sandwich assay using an anti-ESM-1 monoclonal antibody as one antibody and a polyclonal anti-ESM-1 antibody as the other is useful in testing samples for ESM-1 activity.

The foregoing are merely exemplary diagnostic assays for ESM-1 and antibodies. Other methods now or hereafter developed for the determination of these analytes are included within the scope hereof, including the bioassays described above.

Cell Migration Assays

The in vitro model for cell migration comprises a first extracellular matrix containing a cell (the cell which will migrate) and a second extracellular matrix in physical contact with the first extracellular matrix. The cell can be any suitable cell, such as a fibroblast (e.g. a dermal skin fibroblast or a subcutaneous skin fibroblast), an endothelial cell, a monocyte/macrophage, or a tumor cell.

The first extracellular matrix simulates a first natural environment in which the cell naturally resides, and the second extracellular matrix simulates a second natural environment into which the cell naturally migrates from the first natural environment. For example, in wound repair the migrating cell may be a skin fibroblast and its first natural environment is collagenous stroma. The skin fibroblast, during wound repair, naturally migrates from the collagenous stroma into a fibrin clot, which fills the wound. Thus, the first natural environment of the skin fibroblast is the collagenous stroma and the second natural environment of the skin fibroblast is the fibrin clot. The first extracellular matrix of the in vitro model is chosen to simulate the collagenous stroma with the skin fibroblasts therein. The second extracellular matrix of the in vitro model is chosen to simulate the fibrin clot. For example, the second extracellular matrix can be a fibrin gel, or an artificial extracellular matrix. It may also be useful to provide other components in the first and/or second extracellular matrix, such as fibronectin or hyaluronic acid.

The in vitro model can be two dimensional by providing the second extracellular matrix coated onto a surface (such as a microtiter plate, a petri dish, etc.), with the first extracellular matrix, which includes the cells physically positioned on the second extracellular matrix. The cells from the first extracellular matrix will “outmigrate” over the surface of the second extracellular matrix. Alternatively, the model can be three-dimensional by surrounding the first extracellular matrix with the second extracellular matrix (see FIG. 1 where the fibrin extracellular matrix is cast as a gel around the collagen gel extracellular matrix). The cells from the first extracellular matrix will “transmigrate” into the second extracellular matrix.

The migration of cells can be monitored or studied using the in vitro model. It should be readily apparent to those skilled in the art that the migration of cells can only be monitored or studied if the cells are detectable. This can be accomplished several ways. In one embodiment, which includes a fibrin gel into which the cells migrate, the fibrin gel is transparent and the cells can be visualized with a light microscope. Alternatively, the cells provided in the first extracellular matrix can be labeled with a detectable marker. Such detectable markers are known in the art, and include, for example, radioactive labels, fluorescent labels, vital dyes (these non-toxic dyes stain living cells), and labels added by molecular manipulation (such as the βgal gene). The movement of the labeled cells from the first extracellular matrix to the second extracellular matrix can thus be monitored.

The in vivo model comprises an animal model having a naturally occurring first extracellular matrix containing a cell, and a second extracellular matrix in physical contact with the first extracellular matrix. The cell and first and second extracellular matrices are generally as described above for the in vitro model, except that the first extracellular matrix is part of an animal model. For example, a full thickness skin wound is made in an animal (such as a Yorkshire or Minipig). The collagenous stroma of the animal is the first extracellular matrix of the in vivo model, and the second extracellular matrix is provided as a fibrin gel or some artificial extracellular matrix. It may also be desirable with the in vivo model to provide additional cells (like the cells present in the first extracellular matrix of the animal or cells that are intrinsically different from the cells naturally residing in the animal) between the first extracellular matrix and the second extracellular matrix. Then, the migration of those additional cells can also be monitored to determine cell migration into the second extracellular matrix.

Uses of the models are numerous. The primary uses are for screening substances for their effect on cell migration, and for screening extracellular matrices for their effect on cell migration. The method comprises: providing an in vitro model for cell migration as described above; determining a rate of first migration of the cells from the first extracellular matrix into the second extracellular matrix; adding a substance to the in vitro model; and determining a rate of second migration of the cells from the first extracellular matrix into the second extracellular matrix after addition of the substance, wherein an increase in rate of first migration to rate of second migration indicates that the substance increases cell migration, and wherein a decrease in rate of first migration to rate of second migration indicates that the substance decreases cell migration. As indicated above, the cell migration being monitored can be outmigration (in a two dimensional format where the second extracellular matrix is coated on a surface) or transmigration (in a three dimensional format where the second extracellular matrix is cast as a gel, for example, around the first extracellular matrix). Having initially screened the substance for effect on cell migration using the in vitro model, the substance can then be further screened by providing an in vivo model for cell migration as described above, determining a rate of first migration of the cells from the first extracellular matrix into the second extracellular matrix in the in vivo model; adding the substance to the in vivo model; and determining a rate of second migration of the cells from the first extracellular matrix into the second extracellular matrix after addition of the substance to the in vivo model, wherein an increase in rate of first migration to rate of second migration indicates that the substance increases cell migration, and wherein a decrease in rate of first migration to rate of second migration indicates that the substance decreases cell migration in said in vivo model. The use of the in vitro and in vivo models together to screen substances thus provides two levels of screening. As indicated above, it may be desirable when using the in vivo model to provide a plurality of the cells positioned between the first extracellular matrix and the second extracellular matrix of the in vivo model. The method would then further comprise determining another rate of first migration of the plurality of cells from between the first extracellular matrix and the second extracellular matrix into the second extracellular matrix in the in vivo model; and determining another rate of second migration of the plurality of cells from between the first extracellular matrix and the second extracellular matrix into the second extracellular matrix after addition of the substance to the in vivo model, wherein an increase in another rate of first migration to another rate of second migration indicates that the substance increases cell migration, and wherein a decrease in another rate of first migration to another rate of second migration indicates that the substance decreases cell migration in said in vivo model. These additional cells provide an additional means for determining migration of cells into the second extracellular matrix, especially useful when the amount of cells present in the naturally occurring first extracellular matrix are limited or not as easily detected. The additional cells can readily be labeled before they are positioned between the first and second extracellular matrices, and thus can be readily detected. Alternatively, additional cells can be added that are intrinsically different from cells naturally residing in the animal, and detectable based on that difference.

The in vivo model as described above can also be used without the in vitro model to itself screen for substances that affect cell migration. Such a method comprises providing an in vivo model for cell migration as described above, determining a rate of first migration of the cells from the first extracellular matrix into the second extracellular matrix in the in vivo model; adding a substance to the in vivo model; and determining a rate of second migration of the cells from the first extracellular matrix into the second extracellular matrix after addition of the substance to the in vivo model, wherein an increase in rate of first migration to rate of second migration indicates that the substance increases cell migration, and wherein a decrease in rate of first migration to rate of second migration indicates that the substance decreases cell migration in the in vivo model.

Another possibility lies in preliminary screening, which comprises providing a cell and a first and second extracellular matrix, and determining that the cell can move on the first extracellular matrix and the second extracellular matrix. This preliminary screen can precede the screening of substances and/or extracellular matrices with the in vitro or in vivo models, as a means of determining that the cells in question can move on the chosen extracellular matrix. If the cells cannot move on the chosen extracellular matrix, then cell migration will not occur and the method of screening for substances or extracellular matrices that affect cell migration would be meaningless.

Another possibility in migration assays is a method of screening extracellular matrices for the effect of such extracellular matrices on cell migration. The method comprises providing an in vitro model for cell migration as described above; determining a rate of first migration of the cells from the first extracellular matrix into the second extracellular matrix; substituting an artificial extracellular matrix for the second extracellular matrix in the in vitro model; and determining a rate of second migration of the cells from the first extracellular matrix into the artificial extracellular matrix, wherein an increase in rate of first migration to rate of second migration indicates that the artificial extracellular matrix increases cell migration, and wherein a decrease in rate of first migration to rate of second migration indicates that the artificial extracellular matrix decreases cell migration. Preferably, the second extracellular matrix is a fibrin gel when the model is being used to study wound repair.

As with the method of screening substances, the method of screening extracellular matrices can also utilize the in vivo model. Such a method comprises providing an in vivo model for cell migration as described above (again, with the second extracellular matrix preferably being a fibrin gel when the model is being used to study wound repair); determining a rate of first migration of the cells from the first extracellular matrix into the second extracellular matrix; substituting an artificial extracellular matrix for the second extracellular matrix in the in vivo model; and determining a rate of second migration of the cells from the first extracellular matrix into the artificial extracellular matrix, wherein an increase in rate of first migration to rate of second migration indicates that the artificial extracellular matrix increases cell migration, and wherein a decrease in rate of first migration to rate of second migration indicates that the artificial extracellular matrix decreases cell migration.

Antibody

ESM-1 polypeptides can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. A hydropathy plot or similar analyses can be used to identify hydrophilic regions.

An immunogen typically is used to prepare antibodies by immunizing a suitable subject, (e.g., rabbit, goat, mouse or other mammal). An appropriate immunogenic preparation can contain, for example, recombinantly expressed chemically synthesized polypeptide. The preparation can further include an adjuvant, such as Freund's complete or incomplete adjuvant, or similar immunostimulatory agent.

The term “antibody” as used herein refers to immunoglobulin molecules and immunologically active portions of immunoglobulin molecules, i.e., molecules that contain an antigen-binding site, which specifically binds an antigen, such as a polypeptide of the invention. A molecule that specifically binds to a given polypeptide of the invention is a molecule, which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen-binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against an ESM-1 polypeptide can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAPJ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al (1991) Bio Technology 9:1370-1372; Hay et al. (1992) Hum. Antibody Hybridomas 3:81-85; Huse et al. (1989) Science 246:1275-1281; Griffiths et al. (1993) EMBO J. 12:725-734.

ESM-1 Antibody Preparation

Polyclonal antibodies to ESM-1 generally are raised in animals by multiple subcutaneous (sc) or intraperitoneal (ip) injections of ESM-1 and an adjuvant. It may be useful to conjugate ESM-1 or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals ordinarily are immunized against the immunogenic conjugates or derivatives by combining 1 mg or 1 μg of conjugate (for rabbits or mice, respectively) with 3 volumes of Freund's complete adjuvant and injecting the solution intradermally at multiple sites. One month later the animals are boosted with 1/5 to 1/10 the original amount of conjugate in Freund's incomplete adjuvant by subcutaneous injection at multiple sites. 7 to 14 days later animals are bled and the serum is assayed for anti-ESM-1 titer. Animals are boosted until the titer plateaus. Preferably, the animal is boosted with the conjugate of the same ESM-1, but conjugated to a different protein and/or through a different cross-linking agent. Conjugates also can be made in recombinant cell culture as protein fusions. Also, aggregating agents such as alum are used to enhance the immune response. It may be convenient to immunize the animal with an analogous host cell, which has been transformed to express the target receptor of another species.

Monoclonal antibodies are prepared by recovering spleen cells from immunized animals and immortalizing the cells in conventional fashion, e.g. by fusion with myeloma cells or by Epstein-Barr (EB)-virus transformation and screening for clones expressing the desired antibody. The monoclonal antibody preferably does not cross-react with other known ESM-1 polypeptides.

Additionally, chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of an ESM-1 polypeptide. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique it is possible to produce therapeutically useful IgG, IgA, and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Human antibodies can also be produced using various techniques known in the art, including phage display libraries (Homogenous and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(l):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio Technology 10, 779783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Bispecific Antibodies

Bispecific antibodies are monoclonal, preferably human or humanized, antibodies that have binding specificities for at least two different antigens. In the present case, one of the binding specificities is for the PA; the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CHI) containing the site necessary for light-chain binding present in at least one of the fusions. DNAs encoding the immunoglobulin heavy-chain fusions and, if desired, the immunoglobulin light chain, are inserted into separate expression vectors, and are cotransfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers, which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end products such as homodimers.

Bispecific antibodies can be prepared as full-length antibodies or antibody fragments (e.g. F(ab′)2 bispecific antibodies). Techniques for generating bispecific antibodies from antibody fragments have been described in the literature. For example, bispecific antibodies can be prepared can be prepared using chemical linkage. Brennan et al, Science 229:81 (1985) describe a procedure wherein intact antibodies are proteolytic ally cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker, which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared (Tutt et al., J. Immunol. 147:60 (1991)). Exemplary bispecific antibodies can bind to two different epitopes on a given polypeptide herein. Alternatively, an arm can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular a protein of the present invention. Bispecific antibodies can also be used to localize cytotoxic agents to cells, which express a particular a protein of the present invention. These antibodies possess a binding arm to a protein of the present invention and an arm, which binds a cytotoxic agent or a radionuclide chelator, such as EOTUBE, DPTA, DOTA, or TETA. Another bispecific antibody of interest binds the polypeptide and further binds tissue factor (TF).

Pharmaceutical Compositions of Antibodies

Antibodies specifically binding a polypeptide identified herein, as well as other molecules identified by the screening assays disclosed herein, can be administered for the treatment of various disorders in the form of pharmaceutical compositions.

If the polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethyl cellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LLTRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization can be achieved by modifying sulfhydryl residues, lyophilizing from acidic solutions, controlling moisture content, using appropriate additives, and developing specific polymer matrix compositions.

Uses of ESM-1 and its Antibodies

The nucleic acid encoding ESM-1 may be used as a diagnostic for tissue specific typing. For example, such procedures as in situ hybridization, and northern and Southern blotting, and PCR analysis may be used to determine whether DNA and/or RNA encoding ESM-1 are present in the cell type(s) being evaluated.

ESM-1 receptor antibodies are useful in diagnostic assays for ESM-1 expression in specific cells or tissues. The antibodies are labeled in the same fashion as ESM-1 described above and/or are immobilized on an insoluble matrix.

ESM-1 antibodies also are useful for the affinity purification of ESM-1 from recombinant cell culture or natural sources.

Suitable diagnostic assays for ESM-1 and its antibodies are well known per se. Such assays include competitive and sandwich assays, and steric inhibition assays. Competitive and sandwich methods employ a phase-separation step as an integral part of the method while steric inhibition assays are conducted in a single reaction mixture. Fundamentally, the same procedures are used for the assay of ESM-1 and for substances that bind ESM-1, although certain methods will be favored depending upon the molecular weight of the substance being assayed. Therefore, the substance to be tested is referred to herein as an analyte, irrespective of its status otherwise as an antigen or antibody, and proteins that bind to the analyte are denominated binding partners, whether they are antibodies, cell surface receptors, or antigens.

An antibody directed against ESM-1 can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, a-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Assays for Cardiovascular, Endothelial, and Angiogenic Activity

Various assays can be used to test ESM-1 herein for cardiovascular, endothelial, arid angiogenic activity. Such assays include those provided in the Examples below.

Assays for testing for endothelin antagonist activity, as disclosed in U.S. Pat. No. 5,773,414, include a rat heart ventricle binding assay where ESM-1 is tested for its ability to inhibit iodinized endothelin-1 binding in a receptor assay, an endothelin receptor binding assay testing for intact cell binding of radiolabeled endothelin-1 using rabbit renal artery vascular smooth muscle cells, an inositol phosphate accumulation assay where functional activity is determined in Rat-I cells by measuring intra-cellular levels of second messengers, an arachidonic acid release assay that measures the ability of added compounds to reduce endothelin-stimulated arachidonic acid release in cultured vascular smooth muscles, in vitro (isolated vessel) studies using endothelium from male New Zealand rabbits, and in vivo studies using male Sprague-Dawley rats.

Assays for tissue generation activity include, without limitation, those described in WO 95/16035 (bone, cartilage, tendon), WO 95/05846 (nerve, neuronal), and WO 91/07491 (skin, endothelium).

Assays for wound-healing activity include, for example, those described in Winter, Epidermal Wound Healing, Maibach, HI and Rovee, DT, Eds. (Year Book Medical Publishers, Inc., Chicago), pp. 71-112, as modified by the article of Eaglstein and Mertz, J. Invest. Dermatol., 71: 382-384 (1978).

There are several cardiac hypertrophy assays. In vitro assays include induction of spreading of adult rat cardiac myocytes. In this assay, ventricular myocytes are isolated from a single (male Sprague-Dawley) rat, essentially following a modification of the procedure described in detail by Piper et al., “Adult ventricular rat heart muscle cells” in Cell Culture Techniques in Heart and Vessel Research, H. M. Piper, ed. (Berlin: Springer-Verlag, 1990), pp. 36-60. This procedure permits the isolation of adult ventricular myocytes and the long-term culture of these cells in the rod-shaped phenotype. Phenylephrine and Prostaglandin F2 (PGF₂) have been shown to induce a spreading response in these adult cells. The inhibition of myocyte spreading induced by PGF₂ or PGF₂ analogs (e.g., fluprostenol) and phenylephrine by various potential inhibitors of cardiac hypertrophy is then tested.

Assays for Oncological Activity

For cancer, a variety of well-known animal models can be used to further understand the role of ESM-1 in the development and pathogenesis of tumors, and to test the efficacy of candidate therapeutic agents, including antibodies and other antagonists of ESM-1, such as small-molecule antagonists.

The in vivo nature of such models makes them particularly predictive of responses in human patients. Animal models of tumors and cancers (e.g., breast cancer, colon cancer, prostate cancer, lung cancer, etc.) include both non- recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B I O.LP, C17, CM, C57BL, C57, CBA, DBA, DDD, I/st, NC, NFR, NFS, NFS1N, NZB, NZC, NZW, P, RIII, and SJL. In addition, a wide variety of other animals with inherited immunological defects other than the nude mouse have been bred and used as recipients of tumor xenografts. For further details see, e.g., The Nude Mouse in Oncology, Rese E. Boven and B. Winograd, Eds. (CRC Press, Inc., 1991).

The cells introduced into such animals can be derived from known tumor/cancer cell lines, such as any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); or a moderately well differentiated grade II human colon adenocarcinoma cell line, HT-29 (ATCC HTB-38); or from tumors and cancers.

Samples of tumor or cancer cells can be obtained from patients undergoing surgery, using standard conditions involving freezing and storing in liquid nitrogen. Kannali et al., Br. J. Cancer, 48: 689-696 (1983).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. Space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Animal models of breast cancer can be generated, for example, by implanting rat neuroblastoma cells (from which the i7eu oncogene was initially isolated), or neu-transformed NIH-3T3 cells into nude mice, essentially as described by Drebin et al. Proc. Nat. Acad. Sci. USA, 83: 9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54: 4726-4728 (1994) and Too et al. Cancer Research, 55: 681-684 (1995). This model is based on the so-called “METAMOUS5”′ sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated, RNA from pre-passage cells, and cells isolated after one or more rounds of passage analyzed for differential expression of genes of interest. Such passaging techniques can 86 be performed with any known tumor or cancer cell lines. For example, Meth A, CMS4, CMS5, CMS2 1, and WEHI-164 are chemically induced fibrosarcomas of BALB/c female mice (DeLeo et al., J. Exp. Med., 146: 720 (1977)), which provide a highly controllable model system for studying the anti-tumor activities of various agents. Palladino et al., J. Imnmunol., 138: 4023-4032 (1987). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small-cell carcinoma of the lung (SCCL). This tumor can be introduced in normal mice upon injection of tumor fragments from an affected mouse or of cells maintained in culture. Zupi et al., Br. J. Cancer, 41: suppl. 4, 30 (1980). Evidence indicates that tumors can be started from injection of even a single cell and that a very high proportion of infected tumor cells survive. For further information about this tumor model see, Zacharski, Haemostasis, 16: 300-320 (1986).

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals Wu and Sheng Ed. (Basel, 1989), p. 301.

It is noted, however, that necrosis and inflammatory responses following treatment may actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of the ESM-1 gene identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees, and monkeys. Techniques known in the art to introduce a transgene into such animals include pronucleic microinjection (U.S. Pat. No. 4,873,19 1); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer. Lavitrano et al., Cell, 57: 717-73 (1989). For a review, see for example, U.S. Pat. No. 4,736, 866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Nat. Acad. Sci. USA, 89: 6232 636 (1992). The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals can be constructed that have a defective or altered gene encoding ESM-1 identified herein, as a result of homologous recombination between the endogenous gene encoding ESM-1 and altered genomic DNA encoding the same polypeptide introduced into an embryonic cell of the animal. A portion of the genomic DNA encoding ESM-1 can be deleted or replaced with another gene, such as a gene encoding a selectable marker that can be used to monitor integration. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell, 69: 915 (1992). The selected cells are then injected into a blastocyst of an animal (e. g., a mouse or rat) to form aggregation chimeras. See, e.g., Bradley, in Teratocarcinomas and Embryonic Stem Cells: A Practical Approach, E. I Robertson, ed. (IRL: Oxford, 1987), pp. 113-152. A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knockout animals can be characterized, for instance, by their ability to defend against certain pathological conditions and by their development of pathological conditions due to absence of ESM-1.

The efficacy of antibodies specifically binding ESM-1, and other drug candidates, can be tested also in the treatment of spontaneous animal tumors. The data are evaluated for differences in survival, response, and toxicity as compared to control groups. Positive response may require evidence of tumor regression, preferably with improvement of quality of life and/or increased life span.

In addition, spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this -type of tumor in animals.

Aortic ring assays may also be used to test the role of ESM-1 in angiogenesis. Rings of aorta tissue are embedded in gels of collagen, fibrin, or other matrix proteins, followed by outgrowth of branching microvessels (Nicosia and Ottinetti, Lab. Invest. 63:115, 1990). Microvascular growth can be quantitated in the presence and absence of exogenous agents that modulate the expression or function of the target including purified proteins or extracts, conditioned media, antibodies, antisense inhibitors, small molecules, and gene-transducing viral vectors.

Corneal micropocket assays may also be used to test the role of ESM-1 in angiogenesis. Angiogenesis inducing factors or test molecules are introduced into a hydron (or similar material) pellet and surgically implanted into a pocket of the avascular cornea some distance from the limbus. The model may be applied to any of several species including mouse, rat, and rabbit. New blood vessels grow from the limbal vasculature toward the inducer whereupon the corneas can be removed and mounted on microscopic slides for quantitation of a number of variables. Vessel growth can be analyzed in the presence and absence of exogenous agents that modulate the expression or function of the target including purified proteins or extracts, conditioned media, antibodies, antisense inhibitors, small molecules, and gene-transducing viral vectors. Such agents can be administered orally, systemically, or topically.

Matrigel plug assays can also be utilized to test the role of ESM-1 in angiogenesis. Angiogenesis inducing factors or test molecules are introduced into liquid matrigel at 4° C., and then injected subcutaneously into test animals whereupon the matrigel forms a cell-permeable plug into which new blood vessels grow. Vessel ingrowth may be measured directly by histological methods or indirectly by determining hemoglobin content or measurement of endothelial-specific markers. Results are analyzed in the presence and absence of exogenous agents that modulate the expression or function of the target including purified proteins or extracts, conditioned media, antibodies, antisense inhibitors, small molecules, and gene-transducing viral vectors. Such agents can be administered orally, systemically, or locally by suspension in the matrigel implant.

In ovo chick chorioallantoic membrane (CAM) assay can also be utilized to test the role of ESM-1 in angiogenesis. An opening is made in the shell and shell membrane of ten day old embryonated chicken eggs. A sponge, tissue graft, or filter disc containing angiogenesis inducing factors or test substances is placed on the CAM, and the window is sealed with adhesive tape. After several days, neovascular growth is visually observed at the experimental site and can be quantitated by a number of parameters including blood vessel branch points. Results are analyzed in the presence and absence of agents that modulate the expression or function of the target including transfected cell lines or tissues derived from such lines, purified proteins or extracts, conditioned media, antibodies, antisense inhibitors, small molecules, and gene-transducing viral vectors. Such agents can be administered systemically or topically.

Additional tumor growth and angiogenesis assays can also be used to identify the role of ESM-1 in angiogenesis. In one approach, transfected cell lines, which over express wild type, mutated, or antisense genes are analyzed for their ability to affect tumor formation or growth. Such cells may secrete factors that affect endothelial functions or may otherwise exhibit an altered phenotype directly or indirectly affecting neovascularization, as has been previously demonstrated (Im et al., Brit. J. Can. 84:1252, 2001). The degree of angiogenic activity in tumors can be inferred by direct histologic quantitation of microvascular density, by quantitation of tumor-associated endothelial cells, or by quantitation of mRNAs or proteins associated with endothelial expression or with vascular functions. Non-invasive techniques such as positron emission tomography (PET) have also been employed to detect changes in blood flow, blood volume, or vascular permeability in tumors. In a second approach, viral vectors carrying wild type, mutated, or antisense forms of the gene are introduced into tumor-bearing animals by intravenous administration or by direct intratumoral injection, or a combination thereof, thereby allowing transduction of both tumor and vascular elements. This approach has been successfully demonstrated with several genes (Regulier et al., Can. Gene Ther. 8:45, 2001; Kuo et al., PNAS 98:4605, 2001).

Other in vitro and in vivo cardiovascular, endothelial, and angiogenic tests known in the art are also suitable herein.

The results of the cardiovascular, endothelial, and angiogenic study can be further verified by antibody binding studies, in which the ability of anti- ESM-1 antibodies to inhibit the effect of ESM-1 on endothelial cells or other cells used in the cardiovascular, endothelial, and angiogenic assays is tested. Exemplary antibodies include polyclonal, monoclonal, humanized, bispecific, and heteroconjugate antibodies.

Cell-Based Tumor Assays

Cell-based assays and animal models for cardiovascular, endothelial, and angiogenic disorders, such as tumors, can be used to verify the findings of a cardiovascular, endothelial, and angiogenic assay herein, and further to understand the relationship between the genes identified herein and the development and pathogenesis of undesirable cardiovascular, endothelial, and angiogenic cell growth. The role of ESM-1 in the development and pathology of undesirable cardiovascular, endothelial, and angiogenic cell growth, e.g., tumor cells, can be tested by using cells or cells lines that have been identified as being stimulated or inhibited by ESM-1.

In a different approach, cells of a cell type known to be involved in a particular cardiovascular, endothelial, and angiogenic disorder are transfected with ESM-1, and the ability of ESM-1 to induce excessive growth or inhibit growth is analyzed. If the cardiovascular, endothelial, and angiogenic disorder is cancer, suitable tumor cells include, for example, stable tumor cell lines such as the B 104-1-1 cell line (stable NIH-3T3 cell line transfected with the neu protooncogene) and ras-transfected NIH-3T3 cells, which can be transfected with the desired gene and monitored for tumorigenic growth. Such transfected cell lines can then be used to test the ability of poly- or monoclonal antibodies or antibody compositions to inhibit tumorigenic cell growth by exerting cytostatic or cytotoxic activity on the growth of the transformed cells, or by mediating antibody-dependent cellular cytotoxicity (ADCC). Cells transfected with the coding sequences of the genes identified herein can farther be used to identify drug candidates for the treatment of cardiovascular, endothelial, and angiogenic disorders such as cancer.

In addition, primary cultures derived from tumors in transgenic animals (as described above) can be used in the cell-based assays herein, although stable cell lines are preferred. Techniques to derive continuous cell lines from transgenic animals are well known in the art. See, e.g., Small et al., Mol. Cell. Biol., 5: 642-648 (1985).

Screening Assays for Drug Candidates

This invention encompasses methods of screening compounds to identify those that mimic ESM-1 (agonists) or prevent the effect of ESM-1 (antagonists). Screening assays for antagonist candidates are designed to identify compounds that bind or complex with ESM-1, or otherwise interfere with the interaction of ESM-1 with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the candidate with ESM-1 encoded by a nucleic acid identified herein under conditions and for a time sufficient to allow these two components to interact.

In binding assays, the interaction is binding and the complex formed can be isolated or detected in the reaction mixture. In a particular embodiment ESM-1 or the drug candidate is immobilized on a solid phase, e.g., on a microtiter plate, by covalent or non-covalent attachments.

Non-covalent attachment generally is accomplished by coating the solid surface with a solution of ESM-1 and drying. Alternatively, an immobilized antibody, e.g., a monoclonal antibody, specific for ESM-1 to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected.

When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to ESM-1, its interaction with that polypeptide can be assayed by methods well known for detecting protein-protein interactions. Such assays include traditional approaches, such as, e.g., cross-linking, co immunoprecipitation, and co-purification through gradients or chromatographic columns. In addition, protein interactions can be monitored by using a yeast-based genetic system described by Fields and co-workers (Fields and Song, Nature (London), 340: 245-246 (1989); Chien et al., Proc. Nat. Acad. Sci. USA, 88: 9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, and the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL I-lacZ reporter gene under control of a GAL4-activated promoter depends on reconstitution of GAL4 activity via protein-protein interaction. Colonies containing interacting polypeptides are detected with a chromogenic substrate for P-galactosidase. A complete kit (MATCHMAKER) for identifying protein-protein interactions between two specific proteins using the two-hybrid technique is commercially available from Clontech.

This system can also be extended to map protein domains involved in specific protein interactions as well as to pinpoint amino acid residues that are crucial for these interactions.

Compounds that interfere with the interaction of ESM-1 and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

If ESM-1 has the ability to stimulate the proliferation of endothelial cells in the presence of the co-mitogen ConA, then one example of a screening method takes advantage of this ability. Specifically, in the proliferation assay, human umbilical vein endothelial cells are obtained and cultured in 96-well flat-bottomed culture plates (Costar, Cambridge, Mass.) and supplemented with a reaction mixture appropriate for facilitating proliferation of the cells, the mixture containing Con-A (Calbiochem, La Jolla, Calif.). Con-A and the compound to be screened are added and after incubation at 37° C., cultures are pulsed with 3-H-thymidine and harvested onto glass fiber filters (Cambridge Technology, Watertown, Mass.). Mean 3-(H) thymidine incorporation (cpm) of triplicate cultures is determined using a liquid scintillation counter (Beckman Instruments, Irvine, Calif.). Significant 3-(H) thymidine incorporation indicates stimulation of endothelial cell proliferation.

To assay for antagonists, the assay described above is performed; however, in this assay ESM-1 is added along with the compound to be screened and the ability of the compound to inhibit 3-(H)thymidine incorporation in the presence of ESM-1 indicates that the compound is an antagonist to ESM-1. Alternatively, antagonists may be detected by combining ESM-1 and a-potential antagonist with membrane-bound ESM-1 receptors or recombinant receptors under appropriate conditions for a competitive inhibition assay. ESM-1 can be labeled, such as by radioactivity, such that the number of ESM-1 molecules bound to the receptor can be used to determine the effectiveness of the potential antagonist.

More specific examples of potential antagonists include an oligonucleotide that binds to the fusions of immunoglobulin with ESM-1, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments.

Alternatively, a potential antagonist may be a closely related protein, for example, a mutated form of the ESM-1 that recognizes the receptor but imparts no effect, thereby competitively inhibiting the action of ESM-1.

In another assay for antagonists, mammalian cells or a membrane preparation expressing the receptor would be incubated with the labeled ESM-1 in the presence of the candidate compound. The ability of the compound to enhance or block this interaction could then be measured.

Another potential polypeptide antagonist is an antisense construct prepared using antisense technology, where, for example, the antisense molecule acts to block directly the translation of mRNA (or transcription) by hybridizing to targeted mRNA (or genomic DNA) and preventing protein translation (or mRNA transcription) of a protein of the present invention.

Antisense technology can be used to control gene expression through triple-helix formation or antisense DNA or RNA, both of which methods are based on binding of a polynucleotide to DNA or RNA. For example, the 5′coding portion of the polynucleotide sequence, which encodes the mature polypeptides herein, is used to design an antisense RNA oligonucleotide of from about 10 to 100 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the polypeptide. The antisense RNA oligonucleotide hybridizes to the mRNA in vivo and blocks translation of the mRNA molecule into the polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA can be expressed in vivo to inhibit production of the polypeptide. When antisense DNA is used, oligodeoxyribonucleotides derived from the translation initiation site, e.g., between about −10 and +10 positions of the target gene nucleotide sequence, are preferred.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

Preferably, an antisense oligonucleotide can be, for example, about 5, 10, 15, 20, 25, 30, 35, 40, 45, or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, S-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major grove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies, which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an a-anomeric nucleic acid molecule. An a-anomeric nucleic acid molecule forms specific double-stranded hybrids with complementary RNA in which, contrary to the usual a-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The gene encoding the receptor can be identified by numerous methods known to those of skill in the art, for example, ligand panning, and FACS sorting. Coligan et al., Current Protocols in Immun., 1(2): Chapter 5 (1991). Preferably, expression cloning is employed wherein polyadenylated RNA is prepared from a cell responsive to ESM-1 and a cDNA library created from this RNA is divided into pools and used to transfect COS cells or other cells that are not responsive to ESM-1. Transfected-cells that are grown on glass slides are exposed to the labeled ESM-1. ESM-1 can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, ESM-1 can be photoaffinity linked with cell membrane or extract preparations that express the receptor molecule. Cross-linked material is 96 resolved by PAGE and exposed to X-ray film. The labeled complex containing the receptor can be excised, resolved into peptide fragments, and subjected to protein micro sequencing. The amino acid sequence obtained from micro sequencing would be used to design a set of degenerate oligonucleotide probes to screen a cDNA library to identify the gene encoding the putative receptor.

Types of Cardiovascular, Endothelial, and Angiogenic Disorders to be Treated

ESM-1, or agonists or antagonists thereto, that has activity in the cardiovascular, angiogenic, and endothelial assays described herein, and/or whose gene product has been found to be localized to the cardiovascular system, is likely to have therapeutic uses in a variety of cardiovascular, endothelial, and angiogenic disorders, including systemic disorders that affect vessels, such as diabetes mellitus. Its therapeutic utility could include diseases of the arteries, capillaries, veins, and/or lymphatics. Examples of treatments hereunder include treating muscle wasting disease, treating osteoporosis, aiding in implant fixation to stimulate the growth of cells around the implant and therefore facilitate its attachment to its intended site, increasing IGF stability in tissues or in serum, if applicable, and increasing binding to the IGF receptor (since IGF has been shown in vitro to enhance human marrow erythroid and granulocytic progenitor cell growth).

ESM-1 or agonists or antagonists thereto may also be employed to stimulate erythropoiesis or granulopoiesis, to stimulate wound healing or tissue regeneration and associated therapies concerned with re-growth of tissue, such as connective tissue, skin, bone, cartilage, muscle, lung, or kidney, to promote angiogenesis, to stimulate or inhibit migration of endothelial cells, and to proliferate the growth of vascular smooth muscle and endothelial cell production. The increase in angiogenesis mediated by ESM-1 or agonist would be beneficial to ischemic tissues and to collateral coronary development in the heart subsequent to coronary stenosis.

Antagonists are used to inhibit the action of such polypeptides, for example, to limit the production of excess connective tissue during wound healing or pulmonary fibrosis if ESM-1 promotes such production.

This would include treatment of acute myocardial infarction and heart failure.

Moreover, the present invention provides the treatment of cardiac hypertrophy, regardless of the underlying cause, by administering a therapeutically effective dose of ESM-1, or agonist or antagonist thereto.

If the objective is the treatment of human patients, ESM-1 preferably is recombinant human ESM-1 polypeptide (rhESM-1 polypeptide). The treatment for cardiac hypertrophy can be performed at any of its various stages, which may result from a variety of diverse pathologic conditions, including myocardial infarction, hypertension, hypertrophic cardiomyopathy, and valvular regurgitation. The treatment extends to all stages of the progression of cardiac hypertrophy, with or without structural damage of the heart muscle, regardless of the underlying cardiac disorder.

The decision of whether to use the molecule itself or an agonist thereof for any particular indication, as opposed to an antagonist to the molecule, would depend mainly on whether the molecule herein promotes cardio vascularization, genesis of endothelial cells, or angiogenesis or inhibits these conditions. For example, if the molecule promotes angiogenesis, an antagonist thereof would be useful for treatment of disorders where it is desired to limit or prevent angiogenesis. Examples of such disorders include vascular tumors such as haemangioma, tumor angiogenesis, neovascularization in the retina, choroid, or cornea, associated with diabetic retinopathy or premature infant retinopathy or macular degeneration and proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, sear tissue overproduction, for example, that seen in a keloid that forms after surgery, fibrosis after myocardial infarction, or fibrotic lesions associated with pulmonary fibrosis.

If, however, the molecule inhibits angiogenesis, it would be expected to be used directly for treatment of the above conditions.

On the other hand, if the molecule stimulates angiogenesis it would be used itself (or an agonist thereof) for indications where angiogenesis is desired such as peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair, ischemia reperfusion injury, angina, myocardial infarctions such as acute myocardial infarctions, chronic heart conditions, heart failure such as congestive heart failure, and osteoporosis.

If, however, the molecule inhibits angiogenesis, an antagonist thereof would be used for treatment of those conditions where angiogenesis is desired.

Specific types of diseases are described below, where ESM-1 or agonists or antagonists thereof may serve as useful for vascular-related drug targeting or as therapeutic targets for the treatment or prevention of the disorders.

Atherosclerosis is a disease characterized by accumulation of plaques of intimal thickening in arteries, due to accumulation of lipids, proliferation of smooth muscle cells, and formation of fibrous tissue within the arterial wall. The disease can affect large, medium, and small arteries in any organ. Changes in endothelial and vascular smooth muscle cell function are known to play an important role in modulating the accumulation and regression of these plaques.

Hypertension is characterized by raised vascular pressure in the systemic arterial, pulmonary arterial, or portal venous systems. Elevated pressure may result from or result in impaired endothelial function and/or vascular disease.

Inflammatory vasculitides include giant cell arteritis, Takayasu's arteritis, polyarteritis nodosa (including the microangiopathic form), Kawasaki's disease, microscopic polyarightis, Wegener's granulomatosis, and a variety 101 of infectious-related vascular disorders (including Henoch-Schonlein Prupura). Altered endothelial cell function has been shown to be important in these diseases. Reynaud's disease and Reynaud's phenomenon are characterized by intermittent abnormal impairment of the circulation through the extremities on exposure to cold. Altered endothelial cell function has been shown to be important in this disease.

Aneurysms are saccular or fusiform dilatations of the arterial or venous tree that are associated with altered endothelial cell and/or vascular smooth muscle cells.

Arterial restenosis (restenosis of the arterial wall) may occur following angioplasty as a result of alteration in the function and proliferation of endothelial and vascular smooth muscle cells.

Thrombophlebitis and lymphangitis are inflammatory disorders of veins and lymphatics, respectively, that may result from, and/or in, altered endothelial cell function. Similarly, lymphedema is a condition involving impaired lymphatic vessels resulting from endothelial cell function.

The family of benign and malignant vascular tumors is characterized by abnormal proliferation and growth of cellular elements of the vascular system. For example, lymphangiomas are benign tumors of the lymphatic system that are congenital, often cystic, malformations of the lymphatics that usually occur in newborns.

Cystic tumors tend to grow into the adjacent tissue. Cystic tumors usually occur in the cervical and axillary region. They can also occur in the soft tissue of the extremities. The main symptoms are dilated, sometimes reticular, structured lymphatics and lymphocysts surrounded by connective tissue.

Lymphangiomas are assumed to be caused by improperly connected embryonic lymphatics or their deficiency. The result is impaired local lymph drainage.

Another use for ESM-1 antagonists thereto is in the prevention of tumor angiogenesis, which involves vascularization of a tumor to enable it to growth and/or metastasize. This process is dependent on the growth of new blood vessels. Examples of neoplasms and related conditions that involve tumor angiogenesis include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendrogliorna, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, ESM-1 agonist thereto is expected to be useful in reducing the severity of AMD.

Healing of trauma such as wound healing and tissue repair is also a targeted use for ESM-1 or its agonists. Formation and regression of new blood vessels is essential for tissue healing and repair. This category includes bone, cartilage, tendon, ligament, and/or nerve tissue growth or regeneration, as well as wound healing and tissue repair and replacement, and in the treatment of bums, incisions, and ulcers.

ESM-1 agonist or antagonist thereof that induces cartilage and/or bone growth in circumstances where bone is not normally formed has application in the healing of bone fractures and cartilage damage or defects in humans and other animals. Such a preparation employing ESM-1 or agonist or antagonist thereof may have prophylactic use in closed as well as open fracture reduction and also in the improved fixation of artificial joints. De novo bone formation induced by an osteogenic agent contributes to the repair of congenital, trauma induced, or oncologic, resection-induced craniofacial defects, and also is useful in cosmetic plastic surgery.

ESM-1 or agonists or antagonists thereto may also be useful to promote better or faster closure of non-healing wounds, including without limitation pressure ulcers, ulcers associated with vascular insufficiency, surgical and traumatic wounds, and the like.

It is expected that ESM-1 agonist or antagonist thereto may also exhibit activity for generation or regeneration of other tissues, such as organs (including, for example, pancreas, liver, intestine, kidney, skin, or endothelium), muscle (smooth, skeletal, or cardiac), and vascular (including vascular endothelium) tissue, or for promoting the growth of cells comprising such tissues. Part of the desired effects may be by inhibition or modulation of fibrotic scarring to allow normal tissue to regenerate.

ESM-1 agonist or antagonist thereto may also be useful for gut protection or regeneration and treatment of lung or liver fibrosis, reperfusion injury in various tissues, and conditions resulting from systemic cytokine damage. Also, ESM-1 or agonist or antagonist thereto may be useful for promoting or inhibiting differentiation of tissues described above from precursor tissues or cells, or for inhibiting the growth of tissues described above.

ESM-1 agonist or antagonist thereto may also be used in the treatment of periodontal diseases and in other tooth-repair processes. Such agents may provide an environment to attract bone-forming cells, stimulate growth of bone-forming cells, or induce differentiation of progenitors of bone-forming cells ESM-1 or an agonist or an antagonist thereto may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes, since blood vessels play an important role in the regulation of bone turnover and growth.

Another category of tissue regeneration activity that may be attributable to ESM-1 or agonist or antagonist thereto is tendon/ligament formation. A protein that induces tendon/ligament-like tissue or other tissue formation in circumstances where such tissue is not normally formed has application in the healing of tendon or ligament tears, deformities, and other tendon or ligament defects in humans and other animals. Such a preparation may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of ESM-1 or agonist or antagonist thereto contributes to the repair of congenital, trauma-induced, or other tendon or ligament defects of other origin, and is also useful in cosmetic plastic surgery for attachment or repair of tendons or ligaments. The compositions herein may provide an environment to attract tendon- or ligament-forming cells, stimulate growth of tendon- or ligament-forming cells, induce differentiation of progenitors of tendon- or ligament forming cells, or induce growth of tendon/ligament cells or progenitors ex vivo for return in vivo to effect tissue repair. The compositions herein may also be useful in the treatment of tendinitis, carpal tunnel syndrome, and other tendon or ligament defects. The compositions may also include an appropriate matrix and/or sequestering agent as a carrier as is well known in the art.

ESM-1 or its agonist may also be useful for proliferation of neural cells and for regeneration of nerve and brain tissue, i.e., for the treatment of central and peripheral nervous system disease and neuropathies, as well as mechanical and traumatic disorders, that involve degeneration, death, or trauma to neural cells or nerve tissue. More specifically, ESM-1 or its agonist may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions that may be treated in accordance with the present invention include mechanical and traumatic disorders, such as spinal cord disorders, head trauma, and cerebrovascular diseases such as stroke. Peripheral neuropathies resulting from chemotherapy or other medical therapies may also be treatable using ESM-1 agonist or antagonist thereto.

Ischemia-reperfusion injury is another indication. Endothelial cell dysfunction may be important in both the initiation of, and in regulation of the sequelae of events that occur following ischemia-reperfusion injury.

Rheumatoid arthritis is a further indication. Blood vessel growth and targeting of inflammatory cells through the vasculature is an important component in the pathogenesis of rheumatoid and sero-negative forms of arthritis.

ESM-1 or its agonist or antagonist may also be administered prophylactically to patients with cardiac hypertrophy, to prevent the progression of the condition, and avoid sudden death, including death of asymptomatic patients. Such preventative therapy is particularly warranted in the case of patients diagnosed with massive left ventricular cardiac hypertrophy (a maximal wall thickness of 35 mm. or more in adults, or a comparable value in children), or in instances when the hemodynamic burden on the heart is particularly strong.

ESM-1 or its agonist or antagonist may also be useful in the management of atrial fibrillation, which develops in a substantial portion of patients diagnosed with hypertrophic cardiomyopathy. Further indications include angina, myocardial infarctions such as acute myocardial infarctions, and heart failure such as congestive heart failure. Additional non-neoplastic conditions include psoriasis, diabetic and other proliferative retinopathies including retinopathy of prematurity, retrolental fibroplasia, neovascular glaucoma, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

In view of the above, ESM-1 or agonists or antagonists thereof described herein, which are shown to alter or impact endothelial cell function, proliferation, and/or form, are likely to play an important role in the etiology and pathogenesis of many or all of the disorders noted above, and as such can serve as therapeutic targets to augment or inhibit these processes or for vascular-related drug targeting in these disorders.

Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding an ESM-1 polypeptide is a labeled nucleic acid probe capable of hybridizing to mRNA or genomic DNA encoding an ESM-1 polypeptide. The nucleic acid probe can be, for example, a full-length cDNA, such as the nucleic acid of SEQ ID NO:1, or a portion thereof, such as an oligonucleotide of at least 15, 30, 50, 100, 250 or 500 nucleotides in length and sufficient to specifically hybridize under stringent conditions to a mRNA or genomic DNA encoding a ESM-1 polypeptide. Other suitable probes for use in the diagnostic assays of the invention are described herein.

A preferred agent for detecting an ESM-1 polypeptide is an antibody capable of binding to an ESM-1 polypeptide, preferably an antibody with a detectable label. Antibodies can be polyclonal, or more preferably, monoclonal. An intact antibody, or a fragment thereof (e.g., Fab or F(ab) 2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of an ESM-1 polypeptide include enzyme linked immunosorbent assays (ELISAs), Western blots, immunoprecipitations and immunofluorescence. In vitro techniques for detection of genomic DNA include Southern hybridizations. Furthermore, in vivo techniques for detection of an ESM-1 polypeptide include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a ESM-1 polypeptide or mRNA or genomic DNA encoding a ESM-1 polypeptide, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of an ESM-1 polypeptide (e.g., androgen-independent prostate cancer). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits may also include instruction for observing that the tested subject is suffering from or is at risk of developing a disorder associated with aberrant expression of the polypeptide if the amount of the polypeptide or mRNA encoding the polypeptide is above or below a normal level.

For antibody-based kits, the kit may comprise, for example: (1) a first antibody (e.g., attached to a solid support) which binds to an ESM-1 polypeptide; and, optionally, (2) a second, different antibody which binds to either the polypeptide or the first antibody and is conjugated to a detectable agent.

For oligonucleotide-based kits, the kit may comprise, for example: (1) an oligonucleotide, e.g., a detectably labeled oligonucleotide, which hybridizes to a nucleic acid sequence encoding a ESM-1 polypeptide or (2) a pair of primers useful for amplifying a nucleic acid molecule encoding a ESM-1 polypeptide.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples, which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

Prognostic Assays

The methods described herein can furthermore be utilized as diagnostic or prognostic assays to identify subjects having or at risk of developing a disease or disorder associated with aberrant expression or activity of an ESM-1 polypeptide. For example, the assays described herein, such as the preceding diagnostic assays or the following assays, can be utilized to identify a subject having or at risk of developing a disorder associated with aberrant expression or activity of a ESM-1 polypeptide. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of an ESM-1 polypeptide. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a ESM-1 polypeptide in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of an ESM-1 polypeptide. In preferred embodiments, the methods include detecting, in a sample of cells from the subject, the presence or absence of a genetic lesion or mutation characterized by at least one of an alteration affecting the integrity of a gene encoding the ESM-1 polypeptide, or the mis-expression of the gene encoding the ESM-1 polypeptide. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art, which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high-density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of A mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent that cleaves single-stranded regions of the duplex such as which will exist due to base pair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called ADNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment a probe based on a selected-sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a 'GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology, which depends on selective PCR amplification, may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3 ′end of one primer where, under appropriate conditions, mismatch can prevent or reduce polymerase extension (Prossner (1993) Tiblech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect snatch at the 3′end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a ESM-1 polypeptide.

Furthermore, any cell type or tissue, preferably peripheral blood leukocytes, in which the ESM-1 polypeptide is expressed, may be utilized in the prognostic assays described herein.

Pharmacogenomics

Agents, or modulators which have a stimulatory or inhibitory effect on activity or expression of a ESM-1 polypeptide as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of an ESM-1 polypeptide, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms.

Thus, the activity of an ESM-1 polypeptide, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

Monitoring of Effects During Clinical Trials

Monitoring the influence of agents (e.g., drugs, compounds) on the expression or activity of a ESM-1 polypeptide (e.g., the ability to modulate aberrant cell proliferation and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels, or protein activity, can be monitored in clinical trials of subjects exhibiting increased gene expression, protein levels, or protein activity. In such clinical trials, expression or activity of an ESM-1 polypeptide and preferably, that of other polypeptides that have been implicated in prostate cancer, can be used as markers.

For example, and not by way of limitation, genes, including those of the invention, that are modulated in cells by treatment with an agent (e.g., compound, drug or small molecule), which modulates activity or expression of an ESM-1 polypeptide (e.g., as identified in a screening assay described herein) can be identified. Thus, to study the effect of agents on prostate cancer, e.g., androgen-independent prostate cancer, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample (optionally, in the presence and absence of an androgen); (iii) obtaining one or more post-administration samples from the subject; (iv) detecting the level the of the polypeptide or nucleic acid of the invention in the post-administration samples (optionally, in the presence and absence of an androgen); (v) comparing the level (or androgen inducibility) of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to reduce expression or activity of the polypeptide, i.e., to increase the effectiveness of the agent.

Nucleic Acid Transfer

The currently preferred in vivo nucleic acid transfer techniques include transfection with viral (such as adenovirus, lentivirus, Herpes simplex I virus, or adeno-associated virus (AAV)) or non-viral vectors and lipid-based systems (useful lipids for lipid-mediated transfer of the gene are, for example, DOTMA, DOPE, and DC-Chol; see, e.g., Tonkinson et al., Cancer Investigation, 11M: 54-65 (1996)). The most preferred vectors for use in gene therapy are viruses, most preferably adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding ESM-1 is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of ESM-1 from a host cell in which it is placed. Preferably the signal sequence for this purpose is a mammalian signal sequence, most preferably the native signal sequence for ESM-1. Optionally, the vector construct may also include a signal that directs polyadenylation, as well as one or more restriction sites and a translation termination sequence. By way of example, such vectors will typically include a 5′LTR, a tRNA binding site, a packaging signal, an origin of second-strand DNA synthesis, and an YLTR or a portion thereof. Other vectors can be used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis may be used for targeting and/or to facilitate uptake, e.g., capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414 (1990). For a review of the currently known gene marking and gene therapy protocols, see, Anderson et al., Science, 256: 808-813 (1992). See also WO 93/25673 and the references cited therein.

Suitable gene therapy and methods for making retroviral particles and structural proteins can be found in, e.g., U.S. Pat. No. 5,681,746.

Therapeutic Administration

The therapeutically effective dose of ESM-1 or antagonist thereto will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. If ESM-1 has a narrow host range for the treatment of human patients formulations comprising human ESM-1, native-sequence human ESM-1 are preferred. The clinician will administer ESM-1 until a dosage is reached that achieves the desired effect for treatment of the condition in question. For example, if the objective were the treatment of CHF, the amount would be one that inhibits the progressive cardiac hypertrophy associated with this condition. The progress of this therapy is easily monitored by echocardiography. Similarly, in patients with hypertrophic cardiomyopathy, ESM-1 can be administered on an empirical basis.

Combination Therapies

The effectiveness of ESM-1 or an agonist or antagonist thereof in preventing or treating the disorder in question may be improved by administering the active agent serially or in combination with another agent that is effective for those purposes, either in the same composition or as separate compositions. For example, for treatment of cardiac hypertrophy, ESM-1 therapy can be combined with the administration of inhibitors of known cardiac myocyte hypertrophy factors, e.g., inhibitors of cc-adrenergic agonists such as phenylephrine; endothelin-1 inhibitors such as BOSENTAN™ and MOXONODIN™; inhibitors to CT-I (US Pat. No. 5,679,545); inhibitors to LIF; ACE inhibitors; des- aspartate-angiotensin I inhibitors (U.S. Pat. No. 5,773,415), and angiotensin II inhibitors.

For treatment of cardiac hypertrophy associated with hypertension, ESM-1 can be administered in combination with P-adrenergic receptor blocking agents, e.g., propranolol, timolol, tertalolol, carteolol, nadolol, betaxolol, penbutolol, acetobutolol, atenolol, metoprolol, or carvedilol; ACE inhibitors, e.g., quinapril, captopril, enalapril, ramipril, benazepril, fosinopril, or lisinopril; diuretics, e.g., chlorothiazide, hydrochlorothiazide, hydroflumethiazide, methylchlothiazide, benzthiazide, dichlorphenamide, acetazolamide, or indapamide; and/or calcium channel blockers, e.g., diltiazem, nifedipine, verapamil, or nicardipine. Pharmaceutical compositions comprising the therapeutic agents identified herein by their generic names are commercially available, and are to be administered following the manufacturers' instructions for dosage, administration, adverse effects, contraindications, etc. 119 See, e.z., Physicians' Desk Reference (Medical Economics Data Production Co.: Montvale, N.J., 1997), 51 st Edition. Preferred candidates for combination therapy in the treatment of hypertrophic cardiormyopathy are P-adrenergic-blocking drugs (e.g., propranolol, timolol, tertalolol, carteolol, nadolol, betaxolol, penbutolol, acetobutolol, atenolol, metoprolol, or carvedilol), verapamil, difedipine, or diltiazem. Treatment of hypertrophy associated with high blood pressure may require the use of antihypertensive drug therapy, using calcium channel blockers, e.g., diltiazem, nifedipine, verapamil, or nicardipine; P-adrenergic blocking agents; diuretics, e.g., chlorothiazide, hydrochlorothiazide, hydroflumethiazide, methylchlothiazide, benzthiazide, dichlorphenamide, acetazolamide, or indapamide; and/or ACE-inhibitors, e. g., quinapril, captopril, enalapril, ramipril, benazepril, fosinopril, or lisinopril.

For other indications, ESM-1 or its antagonists may be combined with other agents beneficial to the treatment of the bone and/or cartilage defect, wound, or tissue in question. These agents include various growth factors such as EGF, PDGF, TGF- or TGF-, IGF, FGF, and CTGF.

In addition, ESM-1 or its antagonists used to treat cancer may be combined with cytotoxic, chemotherapeutic, or growth-inhibitory agents as identified above. Also, for cancer treatment, ESM-1 or antagonist thereof is suitably administered serially or in combination with radiological treatments, whether involving irradiation or administration of radioactive substances.

The effective amounts of the therapeutic agents administered in combination with ESM-1 or antagonist thereof will be at the physician's or veterinarian's discretion. Dosage administration and adjustment is done to achieve maximal management of the conditions to be treated. For example, for treating hypertension, these amounts ideally take into account use of diuretics or digitalis, and conditions such as hyper- or hypotension, renal impairment, etc. The dose will additionally depend on such factors as the type of the therapeutic agent to be used and the specific patient being treated. Typically, the amount employed will be the same dose as that used, if the given therapeutic agent is administered without PA polypeptide.

For treatment of breast carcinoma, ESM-1 or its antagonists can be administered in combination with, but not limited to, Trastuzumab (Herceptin) with chemotherapy, paclitaxel, docetaxel, epirubicin, mitoxantrone, topotecan, capecitabine, vinorelbine, thiotepa, vincristine, vinblastine, carboplatin or cisplatin, plicamycin, anastrozole, letrozole, exemestane, toremifine, or progestins.

For treatment of acute lymphocytic leukemia, ESM-1 or its antagonists can be administered in combination with, but not limited to, doxorubicin, cytarabine, cyclophosphamide, etoposide, teniposide, allopurinol, or autologous bone marrow transplantation.

For treatment of acute myelocytic and myelomonocytic leukemia, ESM-1, or its antagonists can be administered in combination with, but not limited to, gemtuzumab ozogamicin (Mylotarg), mitoxantrone, idarubicin, etoposide, mercaptopurine, thioguanine, azacitidine, amsacrine, methotrexate, doxorubicin, tretinoin, allopurinol, leukapheresis, prednisone, or arsenic trioxide for acute promyelocytic leukemia.

For treatment of chronic myelocytic leukemia, ESM-1 or its antagonists can be administered in combination with, but not limited to, busulfan, mercaptopurine, thioguanine, cytarabine, plicamycin, melphalan, autologous bone marrow transplantation, or allopurinol.

For treatment of chronic lymphocytic leukemia, ESM-1 or its antagonists can be administered in combination with, but not limited to, vincristine, cyclophosphamide, doxorubicin, cladribine (2-chlorodeoxyadenosine; CdA), allogeneic bone marrow transplant, androgens, or allopurinol.

For treatment of multiple myeloma, ESM-1 or its antagonists can be administered in combination with, but not limited to, etoposide, cytarabine, alpha interferon, dexamethasone, or autologous bone marrow transplantation.

For treatment of carcinoma of the lung (small cell and non-small cell), ESM-1 or its antagonists can be administered in combination with, but not limited to, cyclophosphamide, doxorubicin, vincristine, etoposide, mitomycin, ifosfamide, paclitaxel, irinotecan, or radiation therapy.

For treatment of carcinoma of the colon and rectum, ESM-1 or its antagonists can be administered in combination with, but not limited to, capecitabine, methotrexate, mitomycin, carmustine, cisplatin, irinotecan, or floxuridine.

For treatment of carcinoma of the kidney, ESM-1 or its antagonists can be administered in combination with, but not limited to, alpha interferon, progestins, infusional FUDR, or fluorouracil.

For treatment of carcinoma of the prostate, ESM-1 or its antagonists can be administered in combination with, but not limited to, ketoconazole, doxorubicin, aminoglutethimide, progestins, cyclophosphamide, cisplatin, vinblastine, etoposide, suramin, PC-SPES, or estramustine phosphate.

For treatment of melanoma, ESM-1 or its antagonists can be administered in combination with, but not limited to, carmustine, lomustine, melphalan, thiotepa, cisplatin, paclitaxel, tamoxifen, or vincristine.

For treatment of carcinoma of the ovary, ESM-1 or its antagonists can be administered in combination with, but not limited to, docetaxel, doxorubicin, topotecan, cyclophosphamide, doxorubicin, etoposide, or liposomal doxorubicin.

All references, patents, or applications cited herein are incorporated by reference in their entirety as if written herein.

The present invention will be further illustrated by referring to the following examples, which however, are not to be construed as limiting the scope of the present invention.

### EXAMPLES

### Example 1

Microarray Analysis

Fluorescently labeled (Cy3, Cy5) cDNA probes were generated from the RNA samples (Incyte Genomics). Incyte HG microarray chips were analyzed by competitive hybridization (as described by Yue et al., 2001) to each of four sets of 10 individuals afflicted with cancerous tumors (breast, colon, lung or kidney) and were compared to a normal tissue pool constructed from six sudden death individuals. After error correction and normalization, the fold increase or decrease of tumor tissue expression/normal tissue was calculated.

### Example 2

Signal Sequence Analysis

Prediction of signal sequences was performed using the SignalP 2.0 analysis program. SignalP predicts the presence and location of signal peptide cleavage sites in amino acid sequences from different prokaryotic and eukaryotic organisms. The method incorporates a prediction of cleavage sites and a signal peptide/non-signal peptide prediction based on a combination of several artificial neural networks and hidden Markov models (Nielson et al., 1997).

### Example 3

Sequence Analysis

EST gene assembly was performed utilizing the Crossmatch and Phrap programs (Gordon et al., 1998). Sequence comparisons and molecular weight predictions were determined from the GCG Wisconsin sequence analysis package (Womble, 2000). Analysis of genomic sequence was done by masking all known repetitive elements with the Repeatmasker program (Smit, 1999). The human genomic structure of ESM-1 was determined utilizing sequence data from Celera Human Genome Assembly Release R26. This release is comprised of both Celera and public sequence data and represents an estimated 98% total genome coverage and approximately 4.7× genomic sequence depth (Venter et al., 2001). Markov models of the protein database were constructed utilizing SAM 3.2 (Krogh et al., 1994).

### Example 4

Isolation of ESM-1 cDNA

The 555 base pair full length human ESM-1 was cloned from cDNA generated with SuperScript II reagents (Invitrogen, catalog #11904-018) according to manufacturer's protocol. HDMECs served as the source of RNA, which was isolated using a Qiagen RNeasy Miniprep column (Qiagen, catalog #74104) as per manufacturer's instructions, from cells growing in culture. The forward primer used, esm1f1, was ATGAAGAGCGTCTTGCTGCTG (SEQ ID NO:7). Two reverse primers were used, one containing an in-frame stop codon and one without a stop codon. Primer esm1r1, TCAGCGTGGATTTAACCA (SEQ ID NO:8), contains a stop codon, while primer esm1r2, GCGTGGATTTAACCA (SEQ ID NO:9), does not and was designed for in-frame fusion to a myc/HIS tag. Polymerase chain reactions were performed using 1.0 μl cDNA with the following conditions: 1 μl of forward primer at a concentration of 25 μM, 1 μl of either reverse primer esm1r1 at 25 μM or reverse primer esm1r2 at 25 μM, 0.5 μl of Taq, 1.5 μl of 50 mM MgCl₂, 5 μl of 10× PCR buffer (Invitrogen, catalog # 10342-053), 1 μl of 10 mM dNTP mix (Invitrogen, catalog #18427-013), and water to 50 μl. Cycling parameters were: 3 minutes at 94° C. for hot start, followed by 35 cycles of 1 minute at 94° C., 1 minute at 54° C., and 3 minutes at 72° C., with a final extension of 10 minutes at 72° C. Amplification was determined by running 10 μl of the PCR reaction on a 1.5% agarose gel.

Human ESM-1 was ligated into pCRII-Topo vector using 1 μl PCR product and 1 μl of vector, according to manufacturer's instructions (Invitrogen, catalog #K4600-40). Potential clones were digested with restriction enzyme EcoRI to determine the presence of insert and candidates were sequenced.

### Example 5

Generation of Expression Constructs

Human ESM-1, both with and without stop codons, was transferred into pcDNA3.1 (Invitrogen, catalog #V800-20). The ESM-1 constructs cloned into pCRII-Topo were digested with EcoRI and the insert was gel purified using Qiagen gel extraction columns (Qiagen, catalog #28704) according to manufacturer's instructions. The vector pcDNA3.1 was also digested with EcoRI and dephosphorylated with calf intestinal alkaline phosphatase (CIAP) (Promega, catalog #M182A). Vector and insert were ligated at 20° C. and transformed into competent DH5α cells (Invitrogen, catalog #44-0098). Bacteria were grown overnight at 37° C. on LB plates supplemented with ampicillin (Sigma, catalog #A9393). Clones were then picked and grown overnight at 37° C. in LB with ampicillin. DNA from the clones was isolated using Qiagen DNA MiniPrep kit (Qiagen, catalog #27106) and digested with restriction enzyme EcoRI to determine the presence of inserts.

### Example 6

Transfection and Selection of NIH3T3 Cells

NIH3T3 cells were transfected using a CalPhos transfection kit (Clontech Laboratories, Inc., catalog #K2051) following manufacturers instructions and utilizing 4 μg of either ESM-1 cloned into the expression vector pcDNA3.1 with the myc/HIS tag, or expression vector alone. Cells were maintained in DMEM (GibcoBRL, catalog #11995-040) supplemented with 10% heat inactivated FBS (GibcoBRL, catalog #26140-079) and 1% penicillin/streptomycin (GibcoBRL, catalog #15140-122) at 37° C. and 5% CO₂. Cells were selected in the same media, supplemented with 800 μg/ml G418 (GibcoBRL, catalog #11811-031) for approximately two weeks. Clonal cells were then pooled and transfected cells are considered stable.

### Example 7

In vivo Tumor Formation Assay

Cells are harvested with 0.05% trypsin/EDTA, washed in PBS, and resuspended to 1×10⁷ cells/ml in 60% cold matrigel in PBS. Cells are then injected s.c. into the flanks of female CD-1 nu/nu mice at concentration of 1×10⁶ cells per injection with 27 gauge needle. The diameter of s.c. tumors were measured three times a week using venier calipers.

### Example 8

Real Time Quantitative Reverse Transcription Polymerase Chain Reaction

Gene specific oligonucleotide primer pairs and an oligonucleotide probe labeled with a reporter fluorescent dye at the 5′ end and quencher fluorescent dye at the 3′ end were designed using Primer Express software and purchased from Perkin-Elmer Applied Biosystems (Foster, Calif.). The sequence for the primers and probes are: ATCTGCAAAGACTGTCCCTATGG SEQ ID NO:10 (mouse forward primer), TGCCCGACTGGCAATTG SEQ ID NO:11 (mouse reverse primer), AAGTCTCTTTGCATTCCATCCCGAAGGT SEQ ID NO:12 (mouse probe), GTGGACTGCCCTCAACACTGT SEQ ID NO:13 (human forward primer), TCGAGCACTGTCCTCTTGCA SEQ ID NO:14 (human reverse primer), and CAGTGAGTGCAAAAGCAGCCCGC SEQ ID NO:15 (human probe). RNA samples were purchased from BioChain Institute (San Leandro, Calif.) or isolated using Total RNA Isolation Kit (Ambion, Austin, Tex.) or RNeasy Mini Kit (Qiagen). RNA was then amplified (Ambion) or transcribed into cDNA (Applied Biosystems). Reverse transcription PCR is then run with RNA (200 ng) in a 25 μl reaction mixture or cDNA (10 ng) according to the manufacturer's protocols (Applied Biosystems). The thermal cycling conditions for 200 ng of RNA include one cycle at 48° C. for 30 minutes, one cycle at 95° C. for 10 minutes, followed by 40 cycles at 95° C. for 15 seconds and annealing at 60° C. for 1 minute. The thermal cycling conditions for 10 ng of cDNA include one cycle at 95° C. for 10 minutes, followed by 40 cycles at 95° C. for 15 seconds and annealing at 60° C. for 1 minute. All reactions were performed in the ABI prism 7700 Sequence Detection System (Perkin-Elmer Applied Biosystems). Fluorescent signal for the reaction is collected and characterized as cycle thresholds (CT).

### Example 9

Tissue Preparation Fluorescence Activated Cell Sorting of Tie2/GFP Endothelial Cells

Organs or tumors were removed from mice and immediately suspended in Ca/Mg free PBS on ice. Organs included lung, heart, kidney, and liver; other tissues used were muscles, tumor, placenta, and embryo. Tissue was removed from the PBS, placed on a glass slide, minced into approximately 1mm pieces with two scalpels, and resuspended in collagenase (type 2, Worthington Biochemical Corp.) (25 mg/10 ml). Tissue suspensions were placed in wells of 6-well plates, 4 ml/well, 25 ul of 1% DNase (type IV, Sigma) was added per well and the plate was incubated at 37° C. Plates were placed on a shaker set at low speed to keep tissue suspension mixed. After 30 min incubation, the suspensions were triturated about 5 times with a 5 ml pipette. Trituration was repeated every 10-15 min until the tissues were homogenously dissociated into cell suspensions. Total incubation time was 1-1.5 hrs depending on the tissue. The cell suspension was filtered through 70-micron mesh, diluted 2-3 fold with PBS, and centrifuged at 300 g for 7 min. Red cells were lysed in preparations from lung, heart, or kidney, but not from muscle, tumor, placenta, or embryo. For lysis, the cells were resuspended in 1 ml lysis buffer (Stem Cell Technologies), layered immediately on top of 5 ml of fetal calf serum in a 50 ml tube, and centrifuged as before. Cells were resuspended in PBS with 1% BSA (Sigma) and 0.5 mM EDTA (Life Technologies) at a concentration of about 7×10⁶ cells/ml. Cells were passed through a 40-micron filter immediately before sorting. Cells expressing gfp were separated from non-expressing cells on a Vantage SE (Becton Dickinson) fluorescence activated cell sorter. Gates were set using cell preparations made from non-gfp transformed mouse tissues. Cells from Tie-2-gfp mice that had green fluorescence (measured by F1-1 detectors) were sorted in one direction, and non-fluorescent cells in the same preparation were sorted in the other direction. A sample of unsorted cells was also collected to yield three different populations for comparison (unsorted, gfp+ and gfp−) from each tissue examined. Sorted cells were pelleted, resuspended in RLT buffer (Quiagen) at a rate of <5×10⁶ cells/350 ul and frozen at −80° C. prior to preparation of RNA.

### Example 10

In situ Hybridization

Three oligonucleotide sequences from ESM-1 (CCATCCATGCCTGAGACTGTGCGGTAGCAAGTTTCTCCCC, SEQ ID NO:16; GCCATCTCCAGATGCCATGTCATGCTCCGTGAGAGAAACA, SEQ ID NO:17; and CACCAAAAGGATCCTCCCCATTAGAAGGCTGACACCTCAG SEQ ID NO:18) were labeled with ³³P and used as probes for mRNA in-situ hybridization experiments. Hybridized probes were detected with a PhosphorImager (Cyclone, Packard) and the signal luminescence was measured using OptiQuant software (Packard). Detailed analysis was then performed using ArrayVision software from Imaging Research. Factor VIII immunohistochemistry was performed using DAKO clone F8/86 antibody (code M0616). Slides were briefly post-fixed in acetone, and blocked by incubation in hydrogen peroxidase diluted in water. Primary antibody was diluted 1:50, incubated on slides overnight at 4° C., and visualized using standard ABC techniques (Vector Elite, Vector).

Selected References

Aitkenhead, M., Wang, S. J., Nakatsu, M. N., Mestas, J., Heard, C., and Huges, C. C. W. (2002) Microvasc. Res. 63,159-171.

Bechard, D., Meignin, V., Scherpereel, A., Oudin, S., Kervoaze, G., Bertheau, P., Janin, A., Tonnel, A. B., and Lassalle, P. (2000). J. Vasc. Res. 37, 417-425.

Gordon, D., Abajian, C and Green P. (1998). Consed: a graphical tool for sequence finishing. Genome Res. 8: 195-202.

Krogh, A., Brown, M., Mian, I. S., Sjolander, K. and Haussler, D. (1994). Hidden Markov models in computational biology: Applications to protein modeling. J. Mol. Biol. 235:1501-1531.

Kozak, M. (1999). Initiation of translation in prokaryotes and eukaryotes. Gene 234: 187-208.

Lassalle, P., Delnest, Y., Gosset, P., Gras-Masse, H., Wallert, B., and Tonnel, A. B. (1993) Eur. J. Immunol. 23, 796-803.

Lassalle, P., Molet, S., Janin, A., Van der Heyden, J., Tavernier, J., Fiers, W., Devos, R., and Tonnel, A. B. (1996). J. Biol. Chem. 271(34), 20458-20464.

Murphy, M., Pykett, M. J., Hamish, P., Zang, K. D. and George, D. L. (1993). Identification and characterization of genes differentially expressed in meningiomas. Cell Growth Differ. 4: 715-22.

Nielsen, H., Engelbrecht, J., Brunak, S. and von Heijne, G. (1997). Identification of prokaryotic and eukaryotic signal peptides and prediction of their cleavage sites. Protein Engineering 10: 1-6.

Shimasaki, S., Shimonaka, M., Zhang, H. P. and Ling, N. (1991). Identification of five different insulin-like growth factor binding proteins (IGFBPs) from adult rat serum and molecular cloning of a novel IGFBP-5 in rat and human. J. Biol. Chem. 266: 10646-53.

Smit, A. F. (1999). Interspersed repeats and other mementos of transposable elements in mammalian genomes. Curr. Opin. Genet. Dev. 9: 657-663.

Venter, J. C., Adams, M. D., Myers, E. W., Li, P. W., Mural, R. J., Sutton, G. G., Smith, H. O., Yandell, M., Evans, C. A., Holt, R. A. et al. (2001). The sequence of the human genome. Science, 291: 1304-1351.

Womble, D. D. (2000). GCG: The Wisconsin Package of sequence analysis programs. Methods Mol Biol. 132: 3-22.

Yue, H., Eastman, P. S., Wang, B. B., Minor, J., Doctolero, M. H., Nuttall, R. L., Stack, R., Becker, J. W., Montgomery, J. R., Vainer, M. and Johnston. R. (2001). An evaluation of the performance of cDNA microarrays for detecting changes in global mRNA expression. Nucleic Acids Res., 29:41.

What is claimed is:

1. An isolated ESM-1 polypeptide comprising an amino acid sequence selected from the group consisting of:
(a) an amino acid sequence comprising a sequence of SEQ ID NO:2;
(b) a variant of an amino acid sequence comprising a sequence of SEQ ID NO:2, wherein one or more amino acid residues in said variant differs from the amino acid sequence of said mature form, provided that said variant differs in no more than about 30% of amino acid residues from said amino acid sequence;
(c) a secreted mature form of an amino acid sequence of SEQ ID NO:19;
(d) a variant of a mature form of an amino acid sequence of SEQ ID NO:19, wherein one or more amino acid residues in said variant differs from the amino acid sequence of said mature form, provided that said variant differs in no more than about 30%, of the amino acid residues from the amino acid sequence of said mature form; and
(e) a fragment of the amino acid sequence of SEQ ID NO2, or SEQ ID NO:19.

2. The ESM-1 polypeptide of claim 1, wherein said polypeptide comprises an amino acid sequence of a naturally-occurring allelic variant of an amino acid sequence selected from the group consisting of SEQ ID NO:2, and SEQ ID NO:3.

3. The polypeptide of claim 1, wherein the amino acid sequence of said variant comprises a conservative amino acid substitution.

4. An isolated ESM-1 polypeptide comprising an amino acid sequence selected from the group consisting of: SEQ ID NO:2, or SEQ ID NO:3.

5. An isolated nucleic acid molecule comprising a nucleic acid sequence encoding the polypeptide of claim 1.

6. The isolated nucleic acid molecule of claim 1 wherein the nucleic acid sequence comprises a sequence selected from the group consisting of:
a) a nucleic acid sequence capable of hybridizing under stringent conditions, or which would be capable of hybridizing under said conditions but for the degeneracy of the genetic code, to the DNA sequence of SEQ ID NO:1;
b) a nucleic acid sequence having at least about 80% homology to the DNA sequence of SEQ ID NO:1; and
c) a complement of SEQ ID NO:1.

7. The nucleic acid molecule of claim 6, wherein the nucleic acid molecule is SEQ ID NO:1.

8. The nucleic acid molecule of claim 6, wherein said nucleic acid molecule hybridizes under stringent conditions to a nucleotide sequence of SEQ ID NO:1 or a complement of said nucleotide sequence.

9. A vector comprising the nucleic acid molecule of claim 5, 6, 7, or 8.

10. The vector of claim 9, further comprising a promoter operably linked to said nucleic acid molecule.

11. A host cell comprising the vector of claim 10.

12. A method of producing an ESM-1 polypeptide comprising: growing under suitable nutrient conditions, a host cell of claim 11 under conditions that result in the expression of said ESM-1 polypeptide.

13. A microarray comprising the nucleic acid sequence of claim 1.

14. The microarray of claim 13 wherein said nucleic acid sequence comprises the nucleic acid sequence of SEQ ID NO:1.

15. An antibody that immunospecifically-binds to the ESM-1 polypeptide of claim 1.

16. The antibody of claim 15, wherein said antibody is a monoclonal antibody.

17. The antibody of claim 16, wherein said antibody is an antibody fragment.

18. The antibody of claim 17, wherein said antibody fragment is selected from the group consisting of a Fv fragment, a Fab fragment, (Fab)₂ fragment, and a single chain antibody.

19. The antibody of claim 15, 16, 17, or 18, wherein said antibody is an antagonist.

20. The antibody of claim 19 wherein the antibody is a humanized antibody.

21. The antibody of claim 19 wherein the antibody is a fully human antibody.

22. A method of identifying an agent that binds to the ESM-1 polypeptide of claim 1, the method comprising:
(a) contacting said polypeptide with said agent; and
(b) determining whether said agent binds to said polypeptide.

23. A method for identifying an agent that modulates the expression or activity of the ESM-1 polypeptide of claim 1, the method comprising:
(a) providing a cell expressing said polypeptide in an operational manner;
(b) contacting the cell with said agent; and
(c) determining whether the agent modulates expression or activity of said polypeptide,
whereby an alteration in expression or activity of said peptide indicates said agent modulates expression or activity of said polypeptide.

24. A method for modulating the activity of the ESM-1 polypeptide of claim 1, the method comprising: contacting a cell sample expressing the ESM-1 polypeptide of claim 1 with a compound that binds to said polypeptide in an amount sufficient to modulate the activity of the polypeptide.

25. A method of treating or preventing an angiogenesis associated disorder, said method comprising administering to a subject in which such treatment or prevention is desired the ESM-1 polypeptide of claim 1 in an amount sufficient to treat or prevent said angiogenesis associated disorder in said subject.

26. A method of treating or preventing an angiogenesis associated disorder, said method comprising administering to a subject in which such treatment or prevention is desired the antibody of claim 19 in an amount sufficient to treat or prevent said angiogenesis associated disorder in said subject.

27. A pharmaceutical composition comprising the ESM-1 polypeptide of claim 1 and a pharmaceutically acceptable carrier.

28. A pharmaceutical composition comprising the antibody of claim 19 and a pharmaceutically acceptable carrier.

29. A kit comprising the pharmaceutical composition of claim 28.

30. A method of detecting differentially expressed genes correlated with a cancerous state of a mammalian cell, the method comprising the step of detecting at least one differentially expressed gene product in a test sample derived from a cell suspected of being cancerous, where the gene product is encoded by the nucleic acid sequence SEQ ID NO:1, wherein detection of differentially expressed product is correlated with a cancerous state of the cell from which the test sample was derived.

31. A method for detecting the presence of a nucleic acid molecule of claim 6 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

32. A method for monitoring the progression of an angiogenic disorder in a patient, the method comprising:
a) detecting in a patient sample at a first point in time, the expression of a marker, wherein the marker is the ESM-1 polypeptide of claim 1;
b) repeating step a) at a subsequent point in time; and
c) comparing the level of expression detected in steps a) and b), and therefrom monitoring the progression of the angiogenic disorder.

33. A method of assessing the efficacy of a test compound for inhibiting angiogenesis, the method comprising comparing:
a) expression of a marker in a first sample obtained from a patient exposed to the test compound, wherein the marker is the ESM-1 polypeptide of claim 1, and
b) expression of the marker in a second sample obtained from the patient, wherein the sample is not exposed to the test compound, wherein a significantly lower level of expression of the marker in the first sample, relative to the second sample, is an indication that the test compound is efficacious for inhibiting the cancer in the patient.

34. A method of assessing the efficacy of a therapy for inhibiting angiogenesis in a patient, the method comprising comparing:
a) expression of a marker in the first sample obtained from the patient prior to providing at least a portion of the therapy to the patient, wherein the marker is ESM-1 polypeptide of claim 1, and
b) expression of the marker in a second sample obtained from the patient following provision of the portion of the therapy, wherein a significantly lower level of expression of the marker in the second sample, relative to the first sample, is an indication that the therapy is efficacious for inhibiting the cancer in the patient.

35. A method of selecting a composition for inhibiting angiogenesis in a patient, the method comprising:
(a) obtaining a sample comprising cancer cells from the patient;
(b) separately exposing aliquots of the sample in the presence of a plurality of test compositions;
(c) comparing expression of a marker in each of the aliquots, wherein the marker is selected from the group consisting of the markers of SEQ ID NO:2, and SEQ ID NO:3, and
(d) selecting one of the test compositions which alters the level of expression of the marker in the aliquot containing that test composition, relative to other test compositions.

36. An ESM-1 polypeptide antagonist.

37. The antagonist of claim 36 wherein said antagonist is an antisense molecule.

38. A chimeric molecule comprising the ESM-1 polypeptide of claim 1.

39. A transgenic non-human mammal having integrated into its genome a nucleic acid sequence encoding ESM-1 operatively linked to regulatory elements, wherein expression of said coding sequence increases the level of ESM-1 relative to a non-transgenic mammal of the same species, wherein the coding sequence is the nucleic acid of claim 6.

40. The mammal of claim 39, which is a mouse.

41. A transgenic knockout non-human mammal comprising a homozygous disruption in its endogenous ESM-1 gene, wherein said disruption prevents the expression of a functional ESM-1 protein.


SEQUENCE LISTING (19 sequences)

SEQ ID NO: 1 - DNA, 555 bp, Homo sapiens
atgaagagcgtcttgctgctgaccacgctcctcgtgcctgcacacctggtggccgcctggagcaataattatgcggtggactgccctcaacactgtgacagcagtgagtgcaaaagcagcccgcgctgcaagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgaaattccccttcttccaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctga

SEQ ID NO: 2 - protein, 184 aa, Homo sapiens
MKSVLLLTTLLVPAHLVAAWSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR

SEQ ID NO: 3 - protein, 65 aa, Homo sapiens
VDCPQHCDSSECKSSPRCKRTVLDDCGCCRVCAAGRGETCYRTVSGMDGMKCGPGLRCQPSNGED

SEQ ID NO: 4 - protein, 63 aa, Homo sapiens
ARCPPCSPARCPPEPPGCAELVVLDGCGGCCPVCARQEGEPCGVYTPPCAPGGLRCDPPPGEE

SEQ ID NO: 5 - protein, 184 aa, Rattus sp.
MKSLLLVTTLLIPLHLGMAWSAKYAVDCPEHCDNTECRSSLRCKRTVLDDCGCCQVCAAGPGETCYRTVSGMDGVKCGPGLKCHFYSEEDDFGDEFGVCKDCPYGTFGMDCKETCNCQSGICDRVTGRCLDFPFFQYAAAKSPSRTSASQTERDAASGDGNAVREEIGDRNAARPSVMKWLNPR

SEQ ID NO: 6 - protein, 184 aa, Mus musculus
MKSLLLLTTLLVPLHLGMAWSAKYAVDCPEHCDKTECRSSLRCKRTVLDDCGCCQVCAAGPGETCYRTVSGMDGVKCGPGLKCHFYSEEDDFGDEFGICKDCPYGTFGMECKETCNCQSGICDRVTGRCLDFPFFQYAAAKSPSRTSASHTERDSASGDGNAVREEIGEGNAARPSVMKWLNPR

SEQ ID NO: 7 - DNA, 21 bp, Artificial
  human ESM-1 PCR forward primer
atgaagagcgtcttgctgctg

SEQ ID NO: 8 - DNA, 18 bp, artificial
  human ESM-1 PCR forward primer
tcagcgtggatttaacca

SEQ ID NO: 9 - DNA, 15 bp, Artificial
  human ESM-1 PCR reverse primer
gcgtggatttaacca

SEQ ID NO: 10 - DNA, 23 bp, artificial
  mouse ESM-1 probe
atctgcaaagactgtccctatgg

SEQ ID NO: 11 - DNA, 17 bp, Artificial
  Mouse PCR probe
tgcccgactggcaattg

SEQ ID NO: 12 - DNA, 28 bp, Artificial
  MOUSE PROBE
aagtctctttgcattccatcccgaaggt

SEQ ID NO: 13 - DNA, 21 bp, Artificial
  HUMAN PROBE
gtggactgccctcaacactgt

SEQ ID NO: 14 - DNA, 20 bp, Artificial
  HUMAN PROBE
tcgagcactgtcctcttgca

SEQ ID NO: 15 - DNA, 23 bp, Artificial
  HUMAN PROBE
cagtgagtgcaaaagcagcccgc

SEQ ID NO: 16 - DNA, 40 bp, Artificial
  IN SITU PROBE
ccatccatgcctgagactgtgcggtagcaagtttctcccc

SEQ ID NO: 17 - DNA, 40 bp, Artificial
  INSITU PROBE
gccatctccagatgccatgtcatgctccgtgagagaaaca

SEQ ID NO: 18 - DNA, 40 bp, Artificial
  INSITU PROBE
caccaaaaggatcctccccattagaaggctgacacctcag

SEQ ID NO: 19 - protein, 165 aa, Homo sapiens
WSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR